(19) United States
(12) Patent Application Publication
(10) Pub. No.: US 2020/0216773 A1
(43) Pub. Date: Jul. 9, 2020

(54) Ionic Liquid, Lubricant, and Magnetic Recording Medium

(71) Applicant: Dexerials Corporation, Tokyo (JP)

(72) Inventors: Hirofumi Kondo, Tokyo (JP); Kouki Hatsuda, Tokyo (JP); Nobuo Tano, Tokyo (JP); KyungSung Yun, Tokyo (JP)

(21) Appl. No.: 16/677,992

(22) Filed: Nov. 8, 2019

Related U.S. Application Data
(63) Continuation of application No. 15/546,584, filed on Jul. 26, 2017.

(30) Foreign Application Priority Data
Jan. 28, 2015 (JP) 2015-014398
May 14, 2015 (JP) 2015-099096
Jul. 24, 2015 (JP) 2015-146716

(51) Int. Cl. C10M 105/72; G11B 5/725; C10M 105/70; C07D 471/04; C07D 285/15; C07D 233/58; C07D 207/06; C07C 311/48; C07C 309/06; C07C 255/05; C07C 211/63; C10M 171/00

(52) U.S. Cl. CPC C10M 105/72; C10M 2215/221; C10M 105/70; C07D 471/04; C07D 285/15; C07D 233/58; C07D 207/06; C07C 311/48; C07C 309/06; C07C 255/05; C07C 211/63; C10M 171/00; C10N 2240/204; C10M 2219/104; C10M 2219/09; C10M 2219/0406; C10M 2215/305; C10M 2215/16; C10M 2215/041; C10N 2220/04; C10M 2215/223; C10N 2230/06; C10M 2215/2245; C10N 2230/08; G11B 5/725

(57) ABSTRACT

A lubricant, which includes an ionic liquid including a conjugate base and a conjugate acid including 2 or more cations in a molecule of the conjugate acid, wherein the conjugate acid includes a monovalent group including a straight-chain hydrocarbon group having 6 or more carbon atoms, and an acid that is a source of the conjugate base has a pKa in acetonitrile of 10 or less.

### TECHNICAL FIELD

The present invention relates to an ionic liquid having a dication structure, a lubricant including the ionic liquid, and a magnetic recording medium using the lubricant.

### BACKGROUND ART

Conventionally, in a thin film magnetic recording medium, a lubricant is applied onto a surface of a magnetic layer for the purpose of reducing frictions between a magnetic head and the surface of the magnetic recording medium, or reducing abrasion. In order to avoid adhesion, such as sticktion, an actual film thickness of the lubricant is of a molecular order. Accordingly, it is not exaggeration to say that the most important thing for a thin film magnetic recording medium is to select a lubricant giving excellent abrasion resistance in any environment.

During a life of a magnetic recording medium, it is important that a lubricant is present on a surface of the medium without causing desorption, spin-off, and chemical deteriorations. Making the lubricant present on a surface of a medium is more difficult, as the surface of the thin film magnetic recording medium is smoother. This is because the thin film magnetic recording medium does not have an ability of replenishing a lubricant as with a coating-type magnetic recording medium.

In the case where an adhesion force between a lubricant and a protective film disposed at a surface of a magnetic layer is weak, moreover, a film thickness of the lubricant is reduced during heating or sliding hence accelerating abrasion. Therefore, a large amount of the lubricant is required. The large amount of the lubricant becomes the mobile lubricant, and therefore a function of replenishing the lost lubricant can be provided. However, an excessive amount of the lubricant makes the film thickness of the lubricant larger than the surface roughness. Therefore, a problem associated with adhesion is caused, and in a crucial case, sticktion is caused, which is a factor of driving failures. These problems associated with frictions have not been sufficiently solved by conventional perfluoropolyether (PFPE)-based lubricants.

Particularly for a thin film magnetic recording medium having high surface smoothness, a novel lubricant is designed at a molecular level, and synthesized to solve the above-described trade-off. Moreover, there are numbers of reports regarding lubricity of PFPE. As described, lubricants are very important in magnetic recording media.

Chemical structures of typical PFPE-based lubricants are depicted in Table 1.

TABLE 1
X—CF₂(OCF₂CF₂)ₙ(OCF₂)ₘOCF₂—X(0.5 < n/m < 1)
Fomblin-based lubricants

| | |
|---|---|
| Z | X = —OCF₃ |
| Z-DOL | X = —CH₂OH |
| Z-DIAC | X = —COOH |
| Z-Tetraol | X = —CH₂ OCH₂CHCH₂ OH |
| | OH |
| AM2001 | 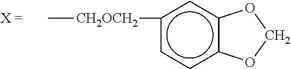 |

Other lubricants

| | |
|---|---|
| A20H | 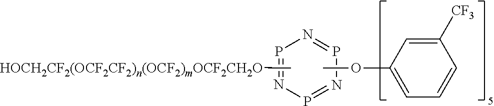 |
| Mono | F—(CF₂CF₂CF₂O)₁—CF₂CF₂CH₂—N(C₃H₇)₂ |

Z-DOL in Table 1 is one of lubricants typically used for thin-film magnetic recording media. Moreover, Z-Tetraol (ZTMD) is a lubricant, in which a functional hydroxyl group is further introduced into a main chain of PFPE, and it has been reported that use of Z-Tetraol enhances reliability of a drive while reducing a space at an interface between a head and a medium. It has been reported that A20H suppresses decomposition of the PFPE main chain with Lewis acid or Lewis base, and improves tribological properties. On the other hand, it has been reported that Mono has a different polymer main chain and different polar groups to those of the PFPE, the polymer main chain and polar groups of Mono are respectively poly-n-propyloxy, and amine, and Mono reduces adhesion interactions at near contact.

However, a typical solid lubricant, which has a high melting point and is considered thermally stable, disturbs an electromagnetic conversion process that is extremely highly sensitive, and moreover, an abrasion powder scraped by a head is generated on a running track. Therefore, abrasion properties are deteriorated. As described above, the liquid lubricant has mobility that enables to move the adjacent lubricant layer to replenish the lubricant removed due to abrasion by the head. However, the lubricant is span-off from a surface of the disk especially at a high temperature during driving of the disk, because of the mobility of the lubricant, and thus the lubricant is reduced. As a result, a protection function is lost. Accordingly, a lubricant having a high viscosity and low volatility is suitably used, and use of such a lubricant enables to prolong a service life of a disk drive with suppressing an evaporation rate.

Considering the above-described lubricating systems, requirements for a low-friction and low-abrasion lubricant used for thin film magnetic recording media are as follows.

(1) Low volatility.
(2) Low surface tension for a surface filling function.
(3) Interaction between terminal polar groups and a surface of a disk.
(4) High thermal and oxidization stability in order to avoid decomposition or reduction over a service period.
(5) Chemically inactive with metals, glass, and polymers, and no abrasion powder generated by a head or a guide.
(6) No toxicity and no flammability.
(7) Excellent boundary lubricating properties.
(8) Soluble with organic solvents.

Recently, an ionic liquid has been attracted attentions as one of solvents for synthesis of organic or inorganic materials and being friendly to the environments in the fields of electricity storage materials, a separation technology, and a catalyst technology. The ionic liquid is roughly classified as a molten salt having a low melting point. The ionic liquid is typically a molten salt having a melting point of 100° C. or lower, among the above-mentioned molten salts. The important properties of the ionic liquid used as a lubricant are low volatility, inflammability, thermal stability, and an excellent dissolving performance.

For example, abrasion and wear of a surface of a metal or ceramic may be reduced by using a certain ionic liquid compared to a conventional hydrocarbon-based lubricant. For example, there is a report that, in the case where an imidazole cation-based ionic liquid is synthesized by substituting with a fluoroalkyl group, and tetrafluoroboric acid salt or hexafluorophosphoric acid salt of alkyl imidazolium is used for steel, aluminium, copper, single crystal SiO₂, silicon, or sialon ceramics (Si—Al—O—N), tribological properties more excellent than those of cyclic phosphazene (X-1P) or PFPE are exhibited. Moreover, there is a report that an ammonium-based ionic liquid reduces frictions more than a base oil in the region of elastohydrodynamic to boundary lubrication. Moreover, effects of the ionic liquid as an additive for a base oil have been studied, and a chemical or tribochemical reaction of the ionic liquid has been researched to understand lubricating systems. However, there are almost no application examples of the ionic liquid to magnetic recording media that require lubricity properties at a molecular level.

Among ionic liquids, a protic ionic liquid is a collective name of a compound formed by a chemical reaction between Bronsted acid and an equivalent amount of Bronsted base. The research associated with an interaction between carboxylic acid and amine by Kohler et al. reports that a 1:1 complex of carboxylic acid and amine can be formed with a chemical equivalent (see, for example, NPLs 1 and 2). It has been reported that perfluorooctanoic acid alkyl ammonium salt is a protic ionic liquid (PIL), and has a significant effect of reducing frictions of a magnetic recording medium Compared to the above-mentioned Z-DOL (see PTLs 1 and 2, and NPLs 3 to 5).

However, the above-mentioned perfluorocarboxylic acid ammonium salts have weak interaction between a cation and an anion in the reaction represented by the following reaction formula (A). According to Le Chatelier's principle, the equilibrium of the reaction is sifted to the left side at a high temperature, and the perfluorocarboxylic acid ammonium salt becomes a dissociated neutral compound and hence thermal stability is deteriorated. Specifically, protons are transferred at a high temperature, the equilibrium is sifted to neutral substance and dissociation occurs (see, for example, NPL 6).

CₙF₂ₙ₊₁COOH+CₙF₂ₙ₊₁NH₂⇄CₙF₂ₙ₊₁COO⁻H₃N⁺CₙH₂ₙ₊₁ (A)

Meanwhile, the limit of a surface recording density of a hard disk is said to be from 1 Tb/in² to 2.5 Tb/in². Currently, the surface recording density is getting close to the limit, but developments of technology for increasing capacities have been actively conducted with a reduction in particle size of magnetic particles as a premise. As the technology for increasing capacities, there are a reduction in an effective flying height and introduction of Shingle Write (BMP).

Moreover, there is “thermally-assisted magnetic recording (heat assisted magnetic recording)” as the next-generation recording technology. The outline of the thermally-assisted magnetic recording is illustrated in FIG. 1. In FIG. 1, the referential numeral 1 is laser light, the referential numeral 2 is near field light, the referential numeral 3 is a recording head (PMR element), and the referential numeral 4 is a reproducing head (TMR element). The problems of the thermally-assisted magnetic recording include a deterioration of durability due to evaporation or deterioration of a lubricant on a surface of a magnetic layer because a recording area is heated by laser during recording and reproducing. Even though it is a short period, there is a possibility that a thin film magnetic recording medium is exposed to a high temperature, which is 400° C. or higher, in thermally-assisted magnetic recording. Therefore, there are concerns about thermal stability of a lubricant generally used for thin film magnetic recording media, such as Z-DOL and Z-TETRAOL.

A protic ionic liquid forms ions as described above, and is typically a material having high thermal stability. The equilibrium of the protic ionic liquid is represented by Scheme 1 below.

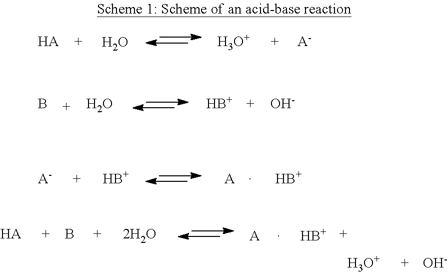

In Scheme 1, HA is Bronsted acid and B is Bronsted base. The acid (HA) and the base (B) are reacted as in Scheme 1 to generate a salt (A⁻HB+).

In Scheme 1, a dissociation constant Kₐ₁ of the acid and a dissociation constant K_{b2} of the base can be represented by Scheme 2 below in the form including the density.

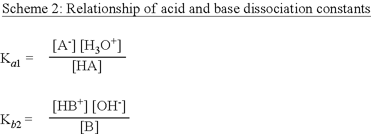

The Kₐ₁ and K_{b2} largely differ depending on a substance. In some cases, the Kₐ₁ and K_{b2} may be large digits, which is inconvenient for handling. Therefore, it is often represented with a negative logarithm. Specifically, the acid dissociation constant is determined as −log₁₀Kₐ₁=pKₐ₁ as represented by Scheme 3 below. Clearly, acidity is stronger, as the pKₐ₁ is smaller.

A difference ΔpKa of the acid dissociation constants of the acid and the base is discussed. The acid-base reaction is influenced by both acidity of the acid and basicity of the base (or acidity of conjugate acid of the base), and the difference ΔpKa in the acidity of the acid and base is represented by Scheme 3 below.

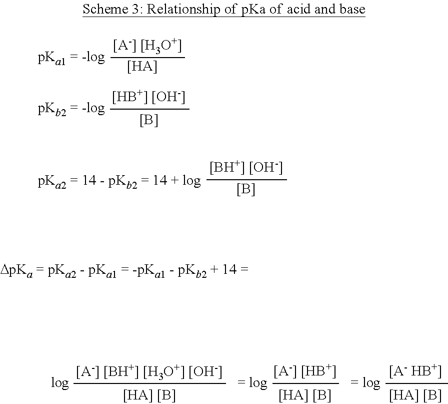

It is indicated that the ΔpKa increases, as the base concentration [A⁻HB⁺] increases relative to the acid concentration and the base concentration.

Meanwhile, Yoshizawa et al. have reported that proton transfer tends to occur when a difference (ΔpKa) of pKa of acid and base is 10 or greater,

[AH]+[B]⇔[A⁻HB⁺]

the equilibrium of the formula above is sifted to the ion side (right side), and stability is enhanced further (see, for example, NPL 6). Moreover, Watanabe et al. has reported that proton transfer and thermal stability of a protic ionic liquid largely depend on ΔpKa, and thermal stability of the ionic liquid is significantly improved by using the acid with which ΔpKa is 15 or greater when DBU (1,8-diazabicyclo[5,4,0]undec-7-ene) is used as a base (see NPL 7). Moreover, Kondo et al. have reported that a perfluorooctanesulfonic acid octadecyl ammonium salt-based protic ionic liquid having large ΔpKa improves durability of a magnetic recording medium (see NPL 8 and PTL 3). In the recent report of Kondo et al. related to thermal resistance of an ionic liquid, it has been reported that a decomposition temperature increases with up to certain degree of ΔpKa, and the decomposition temperature is not increased any further even when ΔpKa is increased from the above-described point (see NPL 9 and NPL 10). Moreover, it is reported that a pyrrolidinium-based ionic liquid including germinal dication may improve thermal resistance more than a typical ionic liquid of monocation (see NPL 11). As described in NPL 11, however, a relationship between a molecular structure constituting the pyrrolidinium-based ionic liquid and physical or chemical characteristics has not been fully understood yet. A combination of a cation and an anion largely influences on physical or chemical characteristics of an ionic liquid. A variety of the anion site is many, but the relationship is not clear unless the cation is a cation structurally similar to the anion (see, for example, NPL 12). For example, viscosity of the liquid increases, as hydrogen bonding strength of halogen is stronger (Cl>Br>I). However, the method for increasing the viscosity is not limited to the increase in the hydrogen bonding strength. For example, the viscosity can be increased by varying an alkyl chain of imidazole. Similarly, the combination of the anion and cation influences on a melting point, surface tension, and thermal stability, but a wide range of researches has not be conducted on an effect of the anion.

Accordingly, it is possible to change physical or chemical characteristics of an ionic liquid by with a combination of cations or anions, but it is difficult to predict.

### CITATION LIST

### Patent Literature

PTL 1: Japanese Patent (JP-B) No. 2581090
PTL 2: JP-B No. 2629725
PTL 3: International Publication No. WO 2014/104342

### Non Patent Literature

NPL 1: Kohler, F., Atrops, H., Kalall, H., Liebermann, E., Wilhelm, E., Ratkovics, F., & Salamon, T., (1981a). Molecular Interactions in Mixtures of Carboxylic acids with amines. 1. Melting Curves and Viscosities, J. Phys. Chem. Vol. 85, No. 17, (August 1981), pp. 2520-2524, ISSN: 0022-3654
NPL 2: Kohler, F., Gopal, R., Goetze, G., Atrops, H., Demiriz, M. A., Liebermann, E., Wilhelm, E., Ratkovics, F., & Palagyl, B., (1981). Molecular Interactions in Mixtures of Carboxylic acids with amines. 2. Volumetric, Conductimetric, and NMR Properties, J. Phys. Chem. Vol. 85, No. 17, pp. 2524-2529, ISSN: 0022-3654
NPL 3: Kondo, H., Seto, J., Haga. S., Ozawa, K., (1989) Novel Lubricants for Magnetic Thin Film Media, Magnetic Soc. Japan, Vol. 13, Suppl. No. S1, pp. 213-218
NPL 4: Kondo, H., Seki, A., Watanabe, H., & Seto, J., (1990). Frictional Properties of Novel Lubricants for Magnetic Thin Film Media, IEEE Trans. Magn. Vol. 26, No. 5, (September 1990), pp. 2691-2693, ISSN: 0018-9464
NPL 5: Kondo, H., Seki, A., & Kita, A., (1994a). Comparison of an Amide and Amine Salt as Friction Modifiers for a Magnetic Thin Film Medium. Tribology Trans. Vol. 37, No. 1, (January 1994), pp. 99-105, ISSN: 0569-8197
NPL 6: Yoshizawa, M., Xu, W., Angell, C. A., Ionic Liquids by Proton Transfer: Vapor pressure, Conductivity, and the Relevance of ΔpKa from Aqueous Solutions, J. Am. Chem. Soc., Vol. 125, pp. 15411-15419 (2003)
NPL 7: Miran, M. S., Kinoshita, H., Yasuda, T., Susan, M. A. B. H., Watanabe, M., Physicochemical Properties Determined by ΔpKa for Protic Ionic Liquids Based on an Organic Super-Strong Base with Various Bronsted Acids, Phys. Chem. Chem. Phys., Vol 14, pp. 5178-5186 (2012)
NPL 8: Hirofumi Kondo, Makiya Ito, Koki Hatsuda, KyungSung Yun and Masayoshi Watanabe, “Novel Ionic Lubricants for Magnetic Thin Film” Media, IEEE TRANSACTIONS ON MAGNETICS, VOL. 49, NO. 7, pp. 3756-3759, Jul. (2013)
NPL 9: Hirofumi Kondo, Makiya Ito, Koki Hatsuda, Nobuo Tano, KyungSung Yun and Masayoshi Watanabe, IEEE International magetic conference Dresden, Germany, May 4-8, 2014
NPL 10: Hirofumi Kondo, Makiya Ito, Koki Hatsuda, Nobuo Tano, KyungSung Yun and Masayoshi Watanabe, IEEE Trans. Magn., 2014, Vol. 50, Issue 11, Article #: 3302504
NPL 11: Anderson, J. L., Ding R., Ellern A., Armstrong D. W., “Structure and Properties of High Stability Geminal Dicationiclonic Liquids”, J. Am. Chem. Soc., 2005, 127, 593-604.
NPL 12: Dzyuba, S. V.; Bartsch, R. A., “Influence of Structural Variations in 1-Alkyl(aralkyl)-3-Methylimidazolium Hexafluorophosphates and Bis(trifluoromethylsulfonyl)imides on Physical Properties of the Ionic Liquids, Chem. Phys. Phys. Chem. 2002, 3, 161-166

### SUMMARY OF INVENTION

### Technical Problem

In the field of magnetic recording media, however, practical properties, such as running performance, antifriction properties, and durability, are not satisfactory due to lack of a performance of a lubricant.

The present invention is proposed based on the above-described conventional situations, and provides an ionic liquid having excellent lubricity even at a high temperature, a lubricant having excellent lubricity even at a high temperature, and a magnetic recording medium having excellent practical properties.

### Solution to Problem

The present inventors have diligently conducted researches. As a result, it has been found that an ionic liquid including a dication can improve thermal stability of the ionic liquid, and friction coefficient is reduced by introducing a long-chain alkyl group to the ionic liquid to significantly improve durability. The present invention has been accomplished based on the above-mentioned findings.

<1> A lubricant including:
an ionic liquid including a conjugate base and a conjugate acid including 2 or more cations in a molecule of the conjugate acid,
wherein the conjugate acid includes a monovalent group including a straight-chain hydrocarbon group having 6 or more carbon atoms, and
an acid that is a source of the conjugate base has a pKa in acetonitrile of 10 or less.
<2> The lubricant according to <1>,
wherein the ionic liquid is represented by General Formula (1) below:

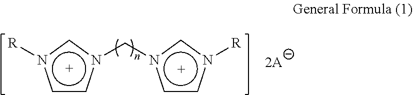

where, in General Formula (1), each R is independently a group including a straight-chain hydrocarbon group having 6 or more carbon atoms, A⁻ is the conjugate base, and n is 1 or greater but 20 or less.
<3> The lubricant according to <1> or <2>,
wherein the ionic liquid is represented by General Formula (1-1) below, General Formula (1-2) below, General Formula (1-3) below, or General Formula (1-4) below:

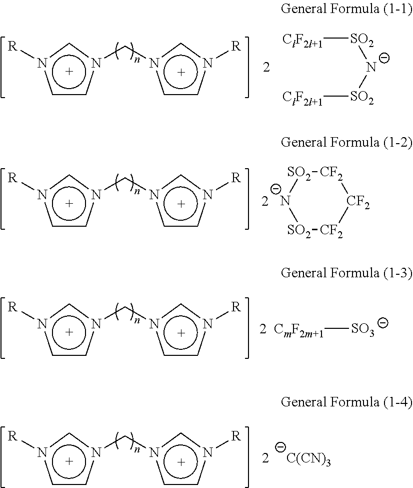

where, in General Formula (1-1), each R is independently a group including a straight-chain hydrocarbon group having 6 or more carbon atoms, n is 1 or greater but 20 or less, and l is 1 or greater but 12 or less,

in General Formula (1-2), each R is independently a group including a straight-chain hydrocarbon group having 6 or more carbon atoms, and n is 1 or greater but 20 or less, in General Formula (1-3), each R is independently a group including a straight-chain hydrocarbon group having 6 or more carbon atoms, n is 1 or greater but 20 or less, and m is 0 or greater but 12 or less, and
in General Formula (1-4), each R is independently a group including a straight-chain hydrocarbon group having 6 or more carbon atoms, and n is 1 or greater but 20 or less.
<4> The lubricant according to <1>,
wherein the ionic liquid is represented by General Formula (2) below:

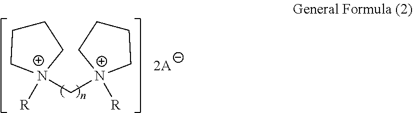

where, in General Formula (2), each R is independently a group including a straight-chain hydrocarbon group having 6 or more carbon atoms, A⁻ is the conjugate base, and n is 1 or greater but 20 or less.
<5> The lubricant according to <1> or <4>,
wherein the ionic liquid is represented by General Formula (2-1) below, General Formula (2-2) below, General Formula (2-3) below, or General Formula (2-4) below:

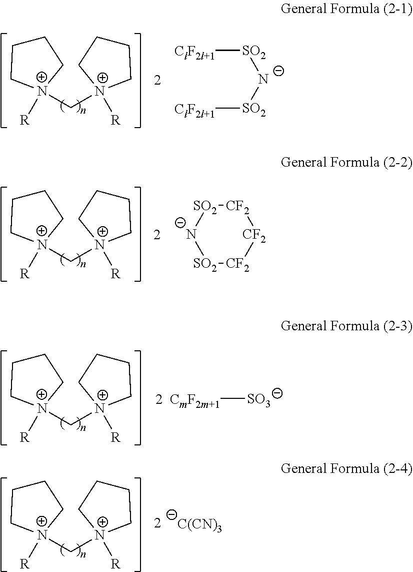

where, in General Formula (2-1), each R is independently a group including a straight-chain hydrocarbon group having 6 or more carbon atoms, n is 1 or greater but 20 or less, and l is 1 or greater but 12 or less,
in General Formula (2-2), each R is independently a group including a straight-chain hydrocarbon group having 6 or more carbon atoms, and n is 1 or greater but 20 or less,
in General Formula (2-3), each R is independently a group including a straight-chain hydrocarbon group having 6 or more carbon atoms, n is 1 or greater but 20 or less, and
m is 0 or greater but 12 or less, and
in General Formula (2-4), each R is independently a group including a straight-chain hydrocarbon group having 6 or more carbon atoms, and n is 1 or greater but 20 or less.
<6> The lubricant according to <1>,
wherein the ionic liquid is represented by General Formula (3) below:

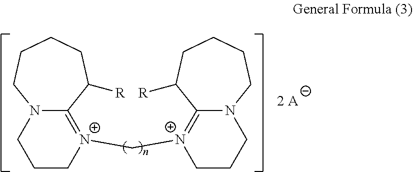

where, in General Formula (3), each R is independently a group including a straight-chain hydrocarbon group having 6 or more carbon atoms, A⁻ is the conjugate base, and n is 1 or greater but 20 or less.
<7> The lubricant according to <1> or <6>,
wherein the ionic liquid is represented by General Formula (3-1) below, General Formula (3-2) below, General Formula (3-3) below, or General Formula (3-4) below:

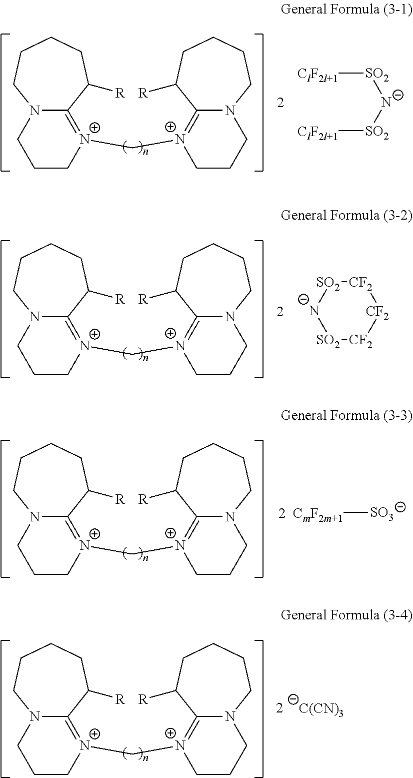

where, in General Formula (3-1), each R is independently a group including a straight-chain hydrocarbon group having 6 or more carbon atoms, n is 1 or greater but 20 or less, and l is 1 or greater but 12 or less,
in General Formula (3-2), each R is independently a group including a straight-chain hydrocarbon group having 6 or more carbon atoms, and n is 1 or greater but 20 or less,
in General Formula (3-3), each R is independently a group including a straight-chain hydrocarbon group having 6 or more carbon atoms, n is 1 or greater but 20 or less, and
m is 0 or greater but 12 or less, and
in General Formula (3-4), each R is independently a group including a straight-chain hydrocarbon group having 6 or more carbon atoms, and n is 1 or greater but 20 or less.
<8> The lubricant according to <1>,
wherein the ionic liquid is represented by General Formula (4) below:

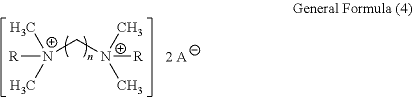

where, in General Formula (4), each R is independently a group including a straight-chain hydrocarbon group having 6 or more carbon atoms, A⁻ is the conjugate base, and n is 1 or greater but 20 or less.
<9> The lubricant according to <1> or <8>,
wherein the ionic liquid is represented by General Formula (4-1) below, General Formula (4-2) below, General Formula (4-3) below, or General Formula (4-4) below:

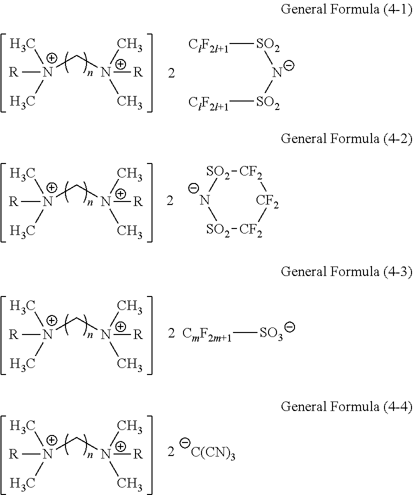

where, in General Formula (4-1), each R is independently a group including a straight-chain hydrocarbon group having 6 or more carbon atoms, n is 1 or greater but 20 or less, and l is 1 or greater but 12 or less,
in General Formula (4-2), each R is independently a group including a straight-chain hydrocarbon group having 6 or more carbon atoms, and n is 1 or greater but 20 or less,
in General Formula (4-3), each R is independently a group including a straight-chain hydrocarbon group having 6 or more carbon atoms, n is 1 or greater but 20 or less, and
m is 0 or greater but 12 or less, and
in General Formula (4-4), each R is independently a group including a straight-chain hydrocarbon group having 6 or more carbon atoms, and n is 1 or greater but 20 or less.
<10> A magnetic recording medium including:
a non-magnetic support;
a magnetic layer disposed on or above the non-magnetic support; and
the lubricant according to any one of <1> to <9> disposed on or above the magnetic layer.
<11> An ionic liquid including:
a conjugate base; and
a conjugate acid including 2 or more cations in a molecule of the conjugate acid,
wherein the conjugate acid includes a monovalent group including a straight-chain hydrocarbon group having 6 or more carbon atoms, and
an acid that is a source of the conjugate base has a pKa in acetonitrile of 10 or less.
<12> The ionic liquid according to <11>,
wherein the ionic liquid is represented by General Formula (1) below:

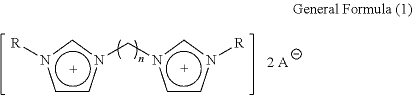

where, in General Formula (1), each R is independently a group including a straight-chain hydrocarbon group having 6 or more carbon atoms, A⁻ is the conjugate base, and n is 1 or greater but 20 or less.
<13> The ionic liquid according to <11> or <12>,
wherein the ionic liquid is represented by General Formula (1-1) below, General Formula (1-2) below, General Formula (1-3) below, or General Formula (1-4) below:

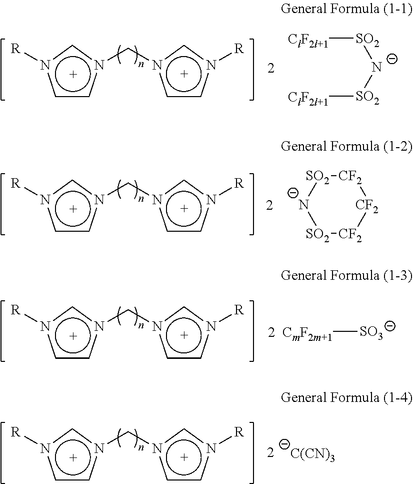

where, in General Formula (1-1), each R is independently a group including a straight-chain hydrocarbon group having 6 or more carbon atoms, n is 1 or greater but 20 or less, and l is 1 or greater but 12 or less,
in General Formula (1-2), each R is independently a group including a straight-chain hydrocarbon group having 6 or more carbon atoms, and n is 1 or greater but 20 or less,
in General Formula (1-3), each R is independently a group including a straight-chain hydrocarbon group having 6 or more carbon atoms, n is 1 or greater but 20 or less, and
m is 0 or greater but 12 or less, and
in General Formula (1-4), each R is independently a group including a straight-chain hydrocarbon group having 6 or more carbon atoms, and n is 1 or greater but 20 or less.
<14> The ionic liquid according to <11>,
wherein the ionic liquid is represented by General Formula (2) below:

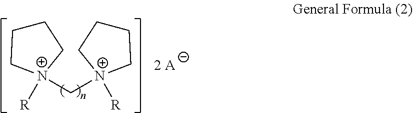

where, in General Formula (2), each R is independently a group including a straight-chain hydrocarbon group having 6 or more carbon atoms, A⁻ is the conjugate base, and n is 1 or greater but 20 or less.
<15> The ionic liquid according to <11> or <14>,
wherein the ionic liquid is represented by General Formula (2-1) below, General Formula (2-2) below, General Formula (2-3) below, or General Formula (2-4) below:

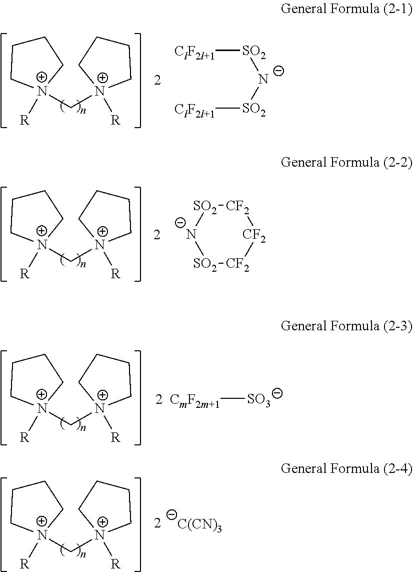

where, in General Formula (2-1), each R is independently a group including a straight-chain hydrocarbon group having 6 or more carbon atoms, n is 1 or greater but 20 or less, and l is 1 or greater but 12 or less,
in General Formula (2-2), each R is independently a group including a straight-chain hydrocarbon group having 6 or more carbon atoms, and n is 1 or greater but 20 or less,
in General Formula (2-3), each R is independently a group including a straight-chain hydrocarbon group having 6 or more carbon atoms, n is 1 or greater but 20 or less, and
m is 0 or greater but 12 or less, and
in General Formula (2-4), each R is independently a group including a straight-chain hydrocarbon group having 6 or more carbon atoms, and n is 1 or greater but 20 or less.
<16> The ionic liquid according to <11>,
wherein the ionic liquid is represented by General Formula (3) below:

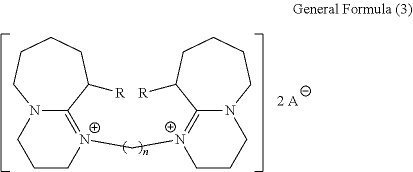

where, in General Formula (3), each R is independently a group including a straight-chain hydrocarbon group having 6 or more carbon atoms, A⁻ is the conjugate base, and n is 1 or greater but 20 or less.
<17> The ionic liquid according to <11> or <16>,
wherein the ionic liquid is represented by General Formula (3-1) below, General Formula (3-2) below, General Formula (3-3) below, or General Formula (3-4) below:

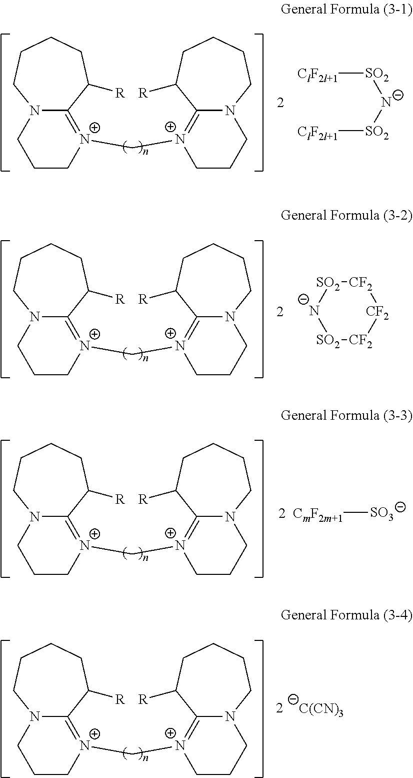

where, in General Formula (3-1), each R is independently a group including a straight-chain hydrocarbon group having 6 or more carbon atoms, n is 1 or greater but 20 or less, and l is 1 or greater but 12 or less,
in General Formula (3-2), each R is independently a group including a straight-chain hydrocarbon group having 6 or more carbon atoms, and n is 1 or greater but 20 or less,
in General Formula (3-3), each R is independently a group including a straight-chain hydrocarbon group having 6 or more carbon atoms, n is 1 or greater but 20 or less, and
m is 0 or greater but 12 or less, and
in General Formula (3-4), each R is independently a group including a straight-chain hydrocarbon group having 6 or more carbon atoms, and n is 1 or greater but 20 or less.
<18> The ionic liquid according to <11>,
wherein the ionic liquid is represented by General Formula (4) below:

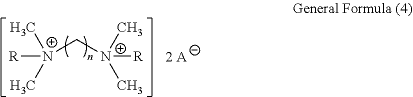

where, in General Formula (4), each R is independently a group including a straight-chain hydrocarbon group having 6 or more carbon atoms, A⁻ is the conjugate base, and n is 1 or greater but 20 or less.
<19> The ionic liquid according to <11> or <18>,
wherein the ionic liquid is represented by General Formula (4-1) below, General Formula (4-2) below, General Formula (4-3) below, or General Formula (4-4) below:

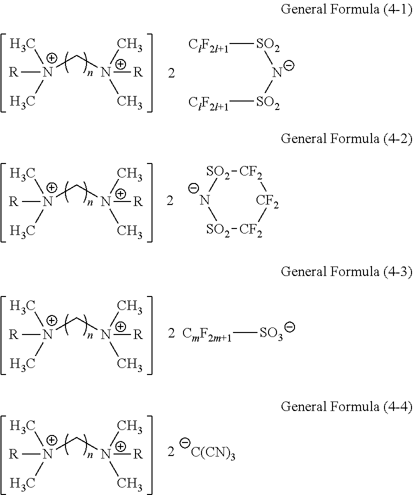

where, in General Formula (4-1), each R is independently a group including a straight-chain hydrocarbon group having 6 or more carbon atoms, n is 1 or greater but 20 or less, and l is 1 or greater but 12 or less,
in General Formula (4-2), each R is independently a group including a straight-chain hydrocarbon group having 6 or more carbon atoms, and n is 1 or greater but 20 or less,
in General Formula (4-3), each R is independently a group including a straight-chain hydrocarbon group having 6 or more carbon atoms, n is 1 or greater but 20 or less, and
m is 0 or greater but 12 or less, and
in General Formula (4-4), each R is independently a group including a straight-chain hydrocarbon group having 6 or more carbon atoms, and n is 1 or greater but 20 or less.

### Advantageous Effects of Invention

According to the present invention, thermal stability of a lubricant, such as evaporation and thermal decomposition, can be improved, and excellent lubricity can be maintained over a long period. In the case where the lubricant is used for a magnetic recording medium, moreover, lubricity is excellent, and practical properties, such as running performances, antifriction properties, and durability, can be improved.

### BRIEF DESCRIPTION OF DRAWINGS

FIG. 1 is a schematic view illustrating thermally-assisted magnetic recording.

FIG. 2 is a cross-sectional view illustrating one example of a hard disk according to one embodiment of the present invention.

FIG. 3 is a cross-sectional view illustrating one example of a magnetic tape according to one embodiment of the present invention.

FIG. 4 is a schematic view illustrating a pin-on-disk test machine.

FIG. 5 depicts the results of the friction test.

FIG. 6 depicts the results of the friction test.

FIG. 7 depicts the results of the friction test.

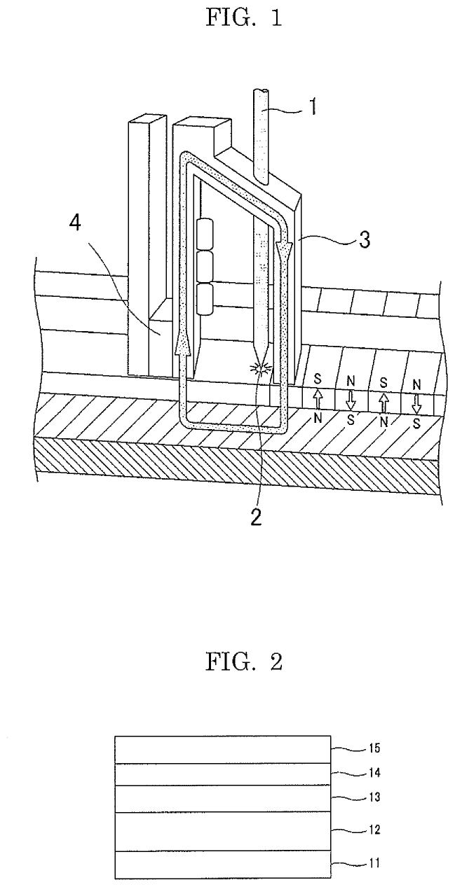

### DESCRIPTION OF EMBODIMENTS

Embodiments of the present invention will be explained in details in the order below with reference to drawings, hereinafter.

1. Lubricant and ionic liquid
2. Magnetic recording medium

### 3. Examples

### 1. Lubricant and Ionic Liquid

A lubricant according to one embodiment of the present invention includes an ionic liquid including a conjugate base and a conjugate acid including 2 or more cations in a molecular of the conjugate acid.

An ionic liquid according to one embodiment of the present invention includes a conjugate base and a conjugate acid including 2 or more cations in a molecular of the conjugate acid.

In the ionic liquid, the conjugate acid includes a monovalent group including a hydrocarbon group. The hydrocarbon group is a straight-chain hydrocarbon group having 6 or more carbon atoms.

In the ionic liquid, an acid that is a source of the conjugate base has a pKa in acetonitrile of 10 or less.

The ionic liquid according to the present embodiment includes a conjugate base and a conjugate acid including 2 or more cations in a molecular of the conjugate acid, where a pKa of an acid that is a base of the conjugate base in acetonitrile is 10 or less, and therefore excellent thermal stability can be exhibited. Moreover, excellent lubricity is obtained in combination with the thermal stability because the cation site includes a monovalent group including a hydrocarbon group having 6 or more carbon atoms.

In the present specification, the pKa is an acid dissociation constant and is an acid dissociation constant in acetonitrile.

The pKa is 10 or less, which is a strong acid, and is preferably 6.0 or less. The lower limit of the pKa is not particularly limited and may be appropriately selected depending on the intended purpose, but the pKa is preferably −5.0 or greater.

Examples of the conjugate base include conjugate bases represented by the following structures.

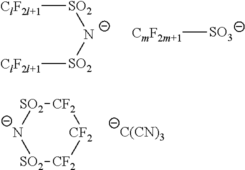

In the conjugate bases, l is 1 or greater but 12 or less and preferably 1 or greater but 6 or less. m is 0 or greater but 12 or less, preferably 1 or greater but 12 or less, and more preferably 1 or greater but 6 or less.

The acid that is a base of the conjugate base (hyaluronic acid: HA) is preferably Bronsted acid regarded as super acid, such as bis((perfluoroalkyl)sulfonyl)imide [(CₗF₂ₗ₊₁SO₂)₂NH] (pKa=0 to 0.3), perfluorocyclopropane sulfoimide (pKa=perfluoroalkyl sulfonic acid (CₘF₂ₘ₊₁SO₃H) (pKa=0.7), tris(perfluoroalkanesulfonyl)methide compounds [(CF₃SO₂)₃CH] (pKa=−3.7), tricyanomethane (pKa=5.1), inorganic acids [e.g., nitric acid (pKa=9.4) and sulfuric acid (pKa=8.7)], tetrafluoroboric acid (pKa=1.8), and hexafluorophosphate, are preferable. For example, the pKa of the above-listed acids are introduced in J. Org. Chem. Vol. 76, No. 2, p. 394.

As the cation, a dication derivative including a monovalent group including a straight-chain hydrocarbon group having 6 or more carbon atoms. Since the hydrocarbon chain is a long chain, a friction coefficient is reduced, and lubricity can be improved.

The number of carbon atoms of the hydrocarbon group in the monovalent group including a straight-chain hydrocarbon group included in the conjugate acid is 6 or greater and preferably 10 or greater.

The upper limit of the number of carbon atoms of the straight-chain hydrocarbon group is not particularly limited and may be appropriately selected depending on the intended purpose. The number of carbon atoms is preferably 25 or less and more preferably 20 or less.

The hydrocarbon group is not limited as long as the hydrocarbon group is in the form of a straight chain, and the hydrocarbon group may be a saturated hydrocarbon group, an unsaturated hydrocarbon group including a double bond at part, or an unsaturated branched hydrocarbon group including a branched chain at part. Among them, an alkyl group that is a saturated hydrocarbon group is preferable in view of antifriction properties. Moreover, the hydrocarbon group is also preferably a straight-chain hydrocarbon group that does not have a branched chain even at part.

For example, the conjugate acid is preferably any of a conjugate acid represented by General Formula (A) below, a conjugate acid represented by General Formula (B) below, a conjugate acid represented by General Formula (C) below, and a conjugate acid represented by General Formula (D) below.

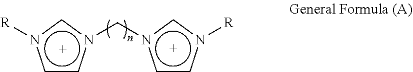

In General Formula (A), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

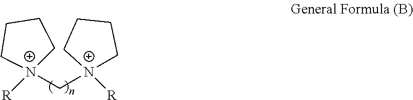

In General Formula (B), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

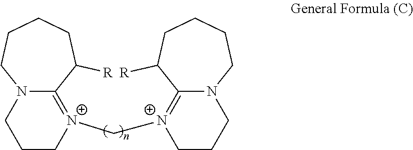

In General Formula (C), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

Note that, the conjugate acid represented by General Formula (C) can have another resonance structure (canonical structure). Specifically, the conjugate acid can have a resonance structure (canonical structure), in which a nitrogen atom at the first position at the bottom of the bicyclic ring is positively charged. In the present invention, the conjugate acid having such a resonance structure (canonical structure) is also included in a conjugate acid represented by General Formula (C).

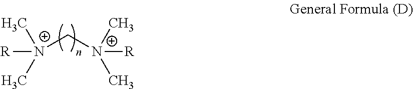

In General Formula (D), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

The upper limit of the number of carbon atoms of the straight-chain hydrocarbon group is not particularly limited and may be appropriately selected depending on the intended purpose. The number of carbon atoms is preferably 25 or less and more preferably 20 or less.

The ionic liquid is preferably an ionic liquid represented by General Formula (1) below, and are more preferably any of an ionic liquid represented by General Formula (1-1) below, an ionic liquid represented by General Formula (1-2) below, an ionic liquid represented by General Formula (1-3) below, and an ionic liquid represented by General Formula (1-4) below.

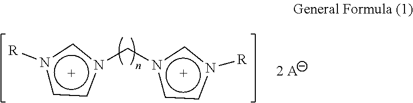

In General Formula (1), each R is independently a group including a straight-chain hydrocarbon group having 6 or more carbon atoms, and is preferably a group including a straight-chain hydrocarbon group having 10 or more carbon atoms. A⁻ is a conjugate base. n is 1 or greater but 20 or less and preferably 3 or greater but 15 or less.

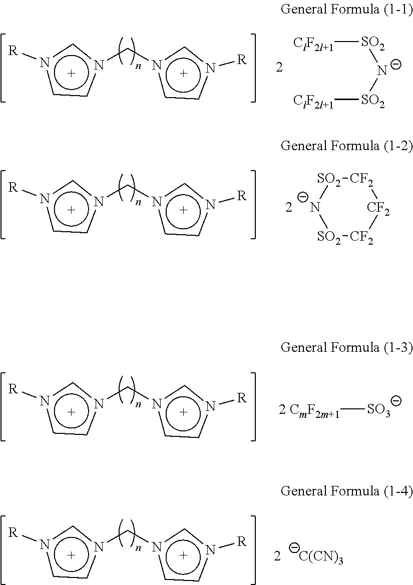

In General Formula (1-1), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. l is 1 or greater but 12 or less and preferably 1 or greater but 6 or less.

In General Formula (1-2), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

In General Formula (1-3), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. m is 0 or greater but 12 or less, preferably 1 or greater but 12 or less, and more preferably 1 or 0.5 greater but 6 or less.

In General Formula (1-4), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

Moreover, the ionic liquid is preferably an ionic liquid represented by General Formula (2) below, and is more preferably any of an ionic liquid represented by General Formula (2-1) below, an ionic liquid represented by General Formula (2-2) below, an ionic liquid represented by General Formula (2-3) below, and an ionic liquid represented by General Formula (2-4) below.

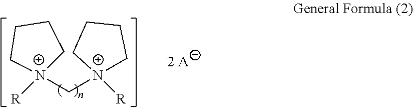

In General Formula (2), each R is independently a group including a straight-chain hydrocarbon group having 6 or more carbon atoms, and is preferably a group including a straight-chain hydrocarbon group having 10 or more carbon atoms. A⁻ is a conjugate base. n is 1 or greater but 20 or less and preferably 3 or greater but 15 or less.

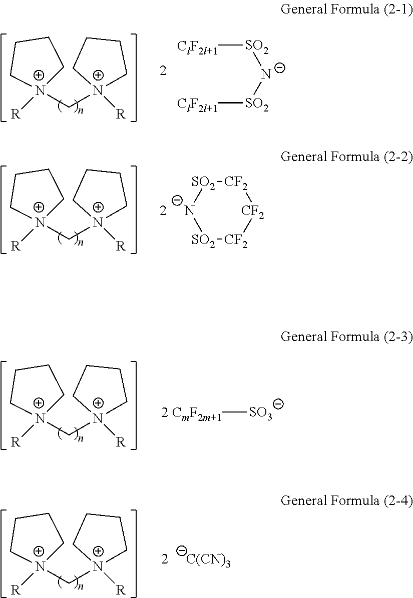

In General Formula (2-1), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. l is 1 or greater but 12 or less and preferably 1 or greater but 6 or less.

In General Formula (2-2), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

In General Formula (2-3), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. m is 0 or greater but 12 or less, preferably 1 or greater but 12 or less, and more preferably 1 or greater but 6 or less.

In General Formula (2-4), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

Moreover, the ionic liquid is preferably an ionic liquid represented by General Formula (3) below, and is more preferably any of an ionic liquid represented by General Formula (3-1) below, an ionic liquid represented by General Formula (3-2) below, an ionic liquid represented by General Formula (3-3) below, and an ionic liquid represented by General Formula (3-4) below.

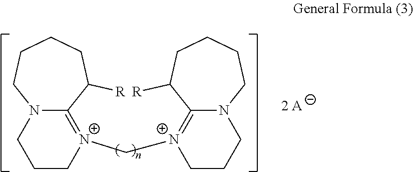

In General Formula (3), each R is independently a group including a straight-chain hydrocarbon group having 6 or more carbon atoms, and is preferably a group including a straight-chain hydrocarbon group having 10 or more carbon atoms. A⁻ is a conjugate base. n is 1 or greater but 20 or less and preferably 3 or greater but 15 or less.

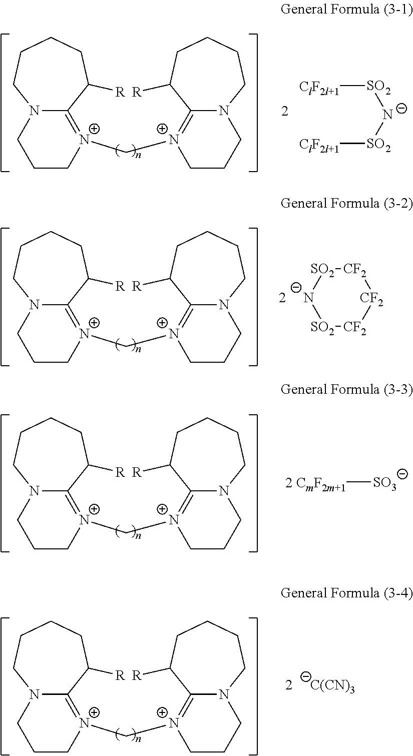

In General Formula (3-1), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. l is 1 or greater but 12 or less and preferably 1 or greater but 6 or less.

In General Formula (3-2), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

In General Formula (3-3), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. m is 0 or greater but 12 or less, preferably 1 or greater but 12 or less, and more preferably 1 or greater but 6 or less.

In General Formula (3-4), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

Moreover, the ionic liquid is preferably an ionic liquid represented by General Formula (4) below, and is more preferably any of an ionic liquid represented by General Formula (4-1) below, an ionic liquid represented by General Formula (4-2) below, an ionic liquid represented by General Formula (4-3) below, and an ionic liquid represented by General Formula (4-4) below.

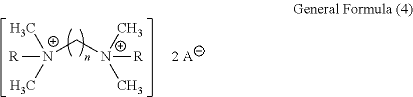

In General Formula (4), each R is independently a group including a straight-chain hydrocarbon group having 6 or more carbon atoms, and is preferably a group including a straight-chain hydrocarbon group having 10 or more carbon atoms. A⁻ is a conjugate base. n is 1 or greater but 20 or less and preferably 3 or greater but 15 or less.

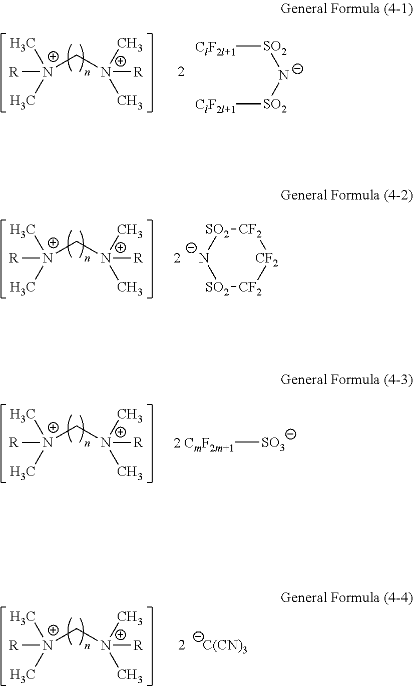

In General Formula (4-1), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. l is 1 or greater but 12 or less and preferably 1 or greater but 6 or less.

In General Formula (4-2), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

In General Formula (4-3), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less. m is 0 or greater but 12 or less, preferably 1 or greater but 12 or less, and more preferably 1 or greater but 6 or less.

In General Formula (4-4), each R is independently a group including a straight-chain hydrocarbon group having 6 or more carbon atoms, and is preferably a group including a straight-chain hydrocarbon group having 10 or more carbon atoms. n is 1 or greater but 20 or less and preferably 3 or greater but 15 or less.

In each of the general formulae above, the upper limit of the number of carbon atoms of the straight-chain hydrocarbon group having 6 or more carbon atoms, which is a preferable embodiment of R, is not particularly limited and may be appropriately selected depending on the intended purpose. The number of carbon atoms is preferably 25 or less and more preferably 20 or less.

A synthesis method of the ionic liquid is not particularly limited and may be appropriately selected depending on the intended purpose. For example, various types of the ionic liquid can be synthesized with reference to a method described in Examples below.

The lubricant in the present embodiment may use the above-described ionic liquid alone, or may be used in combination with a lubricant known in the art. For example, the lubricant can be used in combination with long-chain carboxylic acid, long-chain carboxylic acid ester, perfluoroalkyl carboxylic acid ester, carboxylic acid perfluoroalkyl ester, perfluoroalkyl carboxylic acid perfluoroalkyl ester, a perfluoro polyether derivative.

In order to maintain a lubrication effect under severe conditions, moreover, an extreme-pressure agent may be used in combination at a blending ratio of about 30:70 to 70:30 based on a mass ratio. The extreme-pressure agent performs a function of preventing friction and wear by reacting with a metal surface as a result of friction heat generated when a metal contact is partially formed in a boundary lubrication region, to form a reaction product coating film. As the extreme-pressure agent, for example, any of a phosphorous-based extreme-pressure agent, a sulfur-based extreme-pressure agent, a halogen-based extreme-pressure agent, an organic metal-based extreme-pressure agent, or a complex-based extreme-pressure agent can be used.

Moreover, an anti-rust agent may be used in combination according to the necessity. The anti-rust agent is not particularly limited as long as the anti-rust agent is an anti-rust agent that can be generally used for this type of a magnetic recording medium. Examples of the anti-rust agent include phenols, naphthols, quinones, heterocyclic compounds including a nitrogen atom, heterocyclic compounds including an oxygen atom, and heterocyclic compounds including a sulfur atom. Moreover, the anti-rust agent may be used by blending with a lubricant. Alternatively, the anti-rust agent may be deposited by dividing into 2 or more layers, for example, by forming a magnetic layer on a non-magnetic support, coating the upper part of the magnetic layer with an anti-rust agent layer, followed by coating with a lubricant layer.

As a solvent of the lubricant, moreover, a single or a combination of solvents, for example, alcohol-based solvents, such as isopropyl alcohol (IPA) and ethanol, can be used. For example, a hydrocarbon-based solvent, such as normal hexane, or a fluorine-based solvent may be used by mixing.

### 2. Magnetic Recording Medium

Next, a magnetic recording medium using the above-described lubricant will be explained. The magnetic recording medium presented as one embodiment of the present invention include at least a magnetic layer on or above a non-magnetic support, and the above-described lubricant is deposited on or above the magnetic layer.

The lubricant in the present embodiment can be applied for a so-called a metal thin film magnetic recording medium, in which a magnetic layer is formed on a surface of a non-magnetic support by a method, such as vapor deposition and sputtering. Moreover, the lubricant can be also applied for a magnetic recording medium having a structure where an undercoat layer is disposed between a non-magnetic support and a magnetic layer. Examples of such a magnetic recording medium include magnetic disks and magnetic tapes.

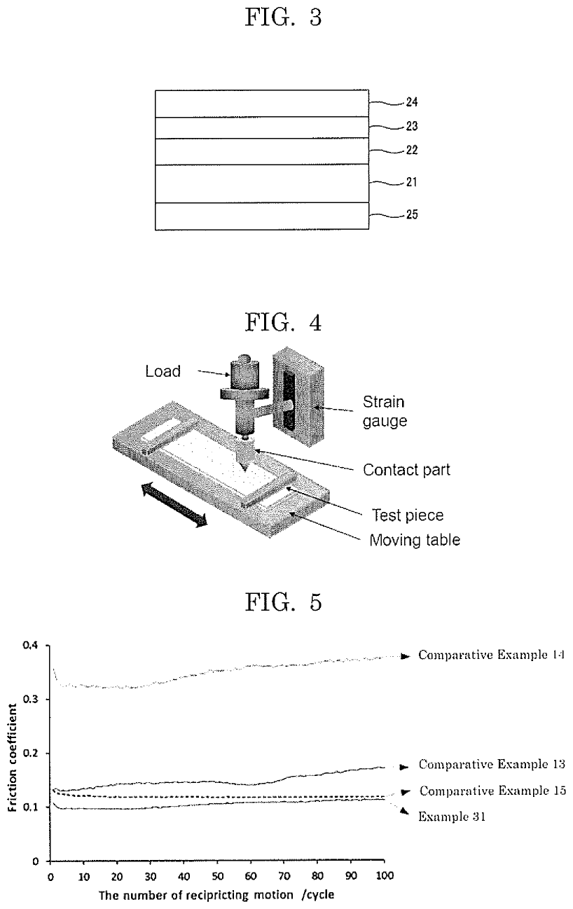

FIG. 2 is a cross-sectional view illustrating one example of a hard disk. The hard disk has a structure where a substrate 11, an undercoat layer 12, a magnetic layer 13, a protective carbon layer 14, and a lubricant layer 15 are sequentially laminated. Moreover, FIG. 3 is a cross-sectional view illustrating one example of a magnetic tape. The magnetic tape has a structure where a back-coating layer 25, a substrate 21, a magnetic layer 22, a protective carbon layer 23, and a lubricant layer 24 are sequentially laminated.

In the magnetic disk illustrated in FIG. 2, the substrate 11 and the undercoat layer 12 are corresponded to a non-magnetic support. In the magnetic tape illustrated in FIG. 3, the substrate 21 is corresponded to a non-magnetic support. When a substrate having rigidity, such as an Al alloy plate and a glass plate, is used as a non-magnetic support, a surface of the substrate may be hardened by forming an oxide film formed by anodizing, or a Ni—P coating on the surface of the substrate

Each of the magnetic layers 13 and 22 is formed as a continuous film by a method, such as plating, sputtering, vacuum deposition, and plasma CVD. Examples of the magnetic layers 13 and 22 include: longitudinal magnetic recording metal magnetic films formed of metals (e.g., Fe, Co, and Ni), Co—Ni-based alloys, Co—Pt-based alloys, Co—Ni—Pt-based alloys, Fe—Co-based alloys, Fe—Ni-based alloys, Fe—Co—Ni-based alloys, Fe—Ni—B-based alloys, Fe—Co—B-based alloys, or Fe—Co—Ni—B-based alloys; and perpendicular magnetic recording metal magnetic thin films, such as Co—Cr-based alloy thin films, and Co—O-based thin films.

In the case where a longitudinal magnetic recording metal magnetic thin film is formed, particularly, a non-magnetic material, such as Bi, Sb, Pb, Sn, Ga, In, Ge, Si, and Tl, is formed as a base layer 12 on a non-magnetic support in advance, and a metal magnetic material is deposited through vapor deposition or sputtering in a perpendicular direction to diffuse the non-magnetic material into the magnetic metal thin film, to thereby improve a coercive force as well as eliminating orientation to assure in-plane isotropy.

Moreover, a hard protective layer 14 or 23, such as a carbon film, a diamond-formed carbon film, a chromium oxide film, and SiO₂ film, may be formed on a surface of the magnetic layer 13 or 22.

Examples of a method for applying the above-mentioned lubricant to such a metal thin film magnetic recording medium include a method for top-coating a surface of the magnetic layer 13 or 22, or a surface of the protective layer 14 or 23 with the lubricant, as illustrated in FIGS. 2 and 3. A coating amount of the lubricant is preferably from 0.1 mg/m² to 100 mg/m², more preferably from 0.5 mg/m² to 30 mg/m², and particularly preferably from 0.5 mg/m² to 20 mg/m².

As illustrated in FIG. 3, moreover, a metal thin film magnetic tape may optionally have a back-coating layer 25, other than a metal magnetic thin film, which is the magnetic layer 22.

The back-coating layer 25 is formed by adding a carbon-based powder for imparting conductivity, or an inorganic pigment for controlling a surface roughness to a resin binder, and applying the resin binder mixture. In the present embodiment, the above-described lubricant may be internally added to the back-coating layer 25, or applied to the back-coating layer 25 as top coating. Moreover, the above-described lubricant may be internally added to both the magnetic layer 22 and the back-coating layer 25, or applied to both the magnetic layer 22 and the back-coating layer 25 as top coating.

As another embodiment, moreover, the lubricant can be applied for a so-called coating-type magnetic recording medium, in which a magnetic coating film is formed as a magnetic layer by applying a magnetic coating material onto a surface of a non-magnetic support. In the coating-type magnetic recording medium, the non-magnetic support, a magnetic powder constituting the magnetic coating film, and the resin binder for use can be selected from any of those known in the art.

Examples of the non-magnetic support include: polymer supports formed by polymer materials, represented by polyesters, polyolefins, cellulose derivatives, vinyl-based resins, polyimides, polyamides, and polycarbonates; metal substrates formed of aluminium alloys, titanium alloys, etc.; ceramic substrates formed of alumina glass, etc.; and glass substrates. Moreover, a shape of the non-magnetic support is not particularly limited, and may be any form, such as a tape, a sheet, and a drum. Moreover, the non-magnetic support may be subjected to a surface treatment by which fine irregularities are formed, in order to control the surface texture of the non-magnetic support.

Examples of the magnetic powder include: ferromagnetic iron oxide-based particles, such as γ-Fe₂O₃, cobalt-coated γ-Fe₂O₃; ferromagnetic chromium dioxide; ferromagnetic metal-based particles formed of a metal, such as Fe, Co, and Ni, or an alloy containing any of the above-listed metals; and hexagonal ferrite particles in the form of hexagonal plates.

Examples of the resin binder include: polymers, such as vinyl chloride, vinyl acetate, vinyl alcohol, vinylidene chloride, acrylic acid ester, methacrylic acid ester, styrene, butadiene, and acrylonitrile; copolymers combining two or more selected from the above-listed polymers; polyurethane resins; polyester resins; and epoxy resins. In order to improve dispersibility of the magnetic powder, a hydrophilic polar group, such as a carboxylic acid group, a carboxyl group, and a phosphoric acid group, may be introduced into any of the above-listed binders.

Other than the magnetic powder and the resin binder, additives, such as a dispersing agent, an abrasive, an antistatic agent, and an anti-rust agent, may be added to the magnetic coating film.

As a method for retaining the above-described lubricant in the coating-type magnetic recording medium, there are a method where the lubricant is internally added to the magnetic layer constituting the magnetic coating film formed on the non-magnetic support, a method where the lubricant is applied on a surface of the magnetic layer as top coating, and a combination of the above-listed methods. In the case where the lubricant is internally added into the magnetic coating film, the lubricant is added in an amount of from 0.2 parts by mass to 20 parts by mass relative to 100 parts by mass of the resin binder.

In the case where a surface of the magnetic layer is top-coated with the lubricant, moreover, a coating amount of the lubricant is preferably from 0.1 mg/m² to 100 mg/m², and more preferably from 0.5 mg/m² to 20 mg/m². As a deposition method in the case where the lubricant is applied as top coating, the ionic liquid is dissolved in a solvent, and the obtained solution may be applied or sprayed, or a magnetic recording medium may be dipped in the solution.

The magnetic recording medium, to which the lubricant of the present embodiment is applied, exhibits excellent running performances, abrasion resistance, and durability because of a lubrication effect, and can further improve thermal stability.

### EXAMPLES

### 3. Examples

Specific examples of the present invention will be explained hereinafter. In the examples, ionic liquids were synthesized, and lubricants each including the ionic liquid were produced. Then, magnetic disks and magnetic tapes were produced using the lubricants, and durability of each disk and durability of each tape were evaluated. Production of a magnetic disk, a durability test of the disk, production of a magnetic tape, and a durability test of the tape were performed in the following manner. Note that, the present invention is not limited to these examples.

### <Production of Magnetic Disk>

A magnetic thin film was formed on a glass substrate to produce a magnetic disk as illustrated in FIG. 2, for example, according to International Publication No. WO2005/068589. Specifically, a chemically reinforced glass disk, which was formed of aluminium silicate glass and had an outer diameter of 65 mm, an inner diameter of 20 mm, and a disk thickness of 0.635 mm, was prepared, and a surface of the glass disk was polished so that Rmax of the surface was to be 4.8 nm, and Ra of the surface was to be 0.43 nm. The glass substrate was subjected to ultrasonic cleaning for 5 minutes each in pure water and in isopropyl alcohol (IPA) having the purity of 99.9% or greater, and the washed glass substrate was left to stand in saturated IPA steam for 1.5 minutes, followed by drying. The resultant glass substrate was provided as a substrate 11.

On the substrate 11, a NiAl alloy (Ni: 50 mol %, Al: 50 mol %) thin film in the thickness of 30 nm as a seed layer, a CrMo alloy (Cr: 80 mol %, Mo: 20 mol %) thin film in the thickness of 8 nm as an undercoat layer 12, and a CoCrPtB alloy (Co: 62 mol %, Cr: 20 mol %, Pt: 12 mol %, B: 6 mol %) thin film in the thickness of 15 nm as a magnetic layer 13 were sequentially formed by DC magnetron sputtering.

Subsequently, a 5 nm-thick protective carbon layer 14 formed of amorphous diamond-like carbon was formed by plasma CVD, and the resultant disk sample was subjected to ultrasonic cleaning for 10 minutes in isopropyl alcohol (IPA) having the purity of 99.9% or greater inside a cleaner to remove impurities on a surface of the disk, followed by drying. Thereafter, an IPA solution of an ionic liquid was applied on a surface of the disk by dip coating in the environment of 25° C. and 50% in relative humidity (RH), to form about 1 nm of a lubricant layer 15.

### <Thermal Stability Measurement>

A TG/DTA measurement was performed by means of EXSTAR6000 available from Seiko Instruments Inc. at a heating rate of 10° C./min and with a temperature range of from 30° C. to 600° C., while introducing air at a flow rate of 200 mL/min.

### <Disk Durability Test 1 (Pin-On-Disk Test)>

By means of a pin-on-disk test machine illustrated in FIG. 4, friction force caused against the arm was detected by a strain gauge under the following measurement conditions.

### [Measurement Conditions]

Load: 15 g
Sliding speed: 1.7 mm/sec (100 mm/min)
Contact area: 3 mm in diameter, steel ball
Sliding distance: 20 mm
Sliding times: 100 times

### <Disk Durability Test 2>

A CSS durability test was performed by means of a commercially available strain-gauge-type disk friction-abrasion tester in the following manner. A hard disk was mounted on a rotatable spindle with tightening torque of 14.7 Ncm. Thereafter, a head slider was attached on the hard disk in a manner that a center of an air bearing surface at the inner circumference side of the head slider relative to the hard disk was 17.5 mm from a center of the hard disk. The head used for the measurement was an IBM3370-type inline head, a material of the slider was Al₂O₃—TiC, and the head load was 63.7 mN. In the test, the maximum value of friction force was monitored per CSS (contact, start, and stop) in the environment of 100 in cleanliness, 25° C., and 60% RH. The number of times when a coefficient of friction was greater than 1.0 was determined as a result of the CSS durability test. When a result of the CSS durability test was greater than 50,000, the result was represented as “>50,000.” Moreover, a CSS durability test was similarly performed after performing a heating test for 3 minutes at a temperature of 300° C., in order to study heat resistance.

### <Production of Magnetic Tape>

A magnetic tape having a cross-sectional structure as illustrated in FIG. 3 was produced. First, Co was deposited on a substrate 21 formed of a 5 μm-thick MICTRON (aromatic polyimide) film available from TORAY INDUSTRIES, INC. by oblique deposition to form a magnetic layer 22 formed of a ferromagnetic metal thin film having a film thickness 100 nm. Next, a carbon protective layer 23 formed of 10 nm-thick diamond-like carbon was formed on a surface of the ferromagnetic metal thin film by plasma CVD, followed by cutting the resultant into a strip having a width of 6 mm. An ionic liquid dissolved in IPA was applied onto the carbon protective layer 23 in a manner that a film thickness of the ionic liquid solution was about 1 nm. In this manner, a lubricant layer 24 is formed to thereby produce a sample tape.

### <Tape Durability Test>

Each sample tape was subjected to a measurement of still durability in an environment having a temperature of −5° C. and in an environment having a temperature of 40° C. and 30% RH, and measurements of a coefficient of friction and shuttle durability in an environment having a temperature of −5° C. and in an environment having a temperature of 40° C. and 90% RH. The still durability was evaluated by a decay time of an output in a paused state decayed by −3 dB. The shuttle resistant was evaluated by the number of shuttles taken until an output was reduced by 3 dB when repeated shuttle run was performed for 2 minutes per time. Moreover, a durability test was similarly performed after performing a heating test for 10 minutes at a temperature of 100° C., in order to study heat resistance.

The ionic liquid according to the present embodiment includes a conjugate base and a conjugate acid including 2 or more cations in a molecule of the conjugate acid, where a pKa of an acid that is a base of the conjugate base in acetonitrile is 10 or less. Moreover, the cation site preferably includes a monovalent group including a hydrocarbon group having 6 or more carbon atoms. Thermal stability of such an ionic liquid, and durability of a magnetic recording medium using the ionic liquid were studied.

### Example 1

### Synthesis of 1,9-bis[1-octadecylimidazolium bis(trifluoromethanesulfonyl)imide]nonane

Synthesis of 1,9-bis[1-octadecylimidazolium bis(trifluoromethanesulfonyl)imide]nonane was performed according to the following scheme.

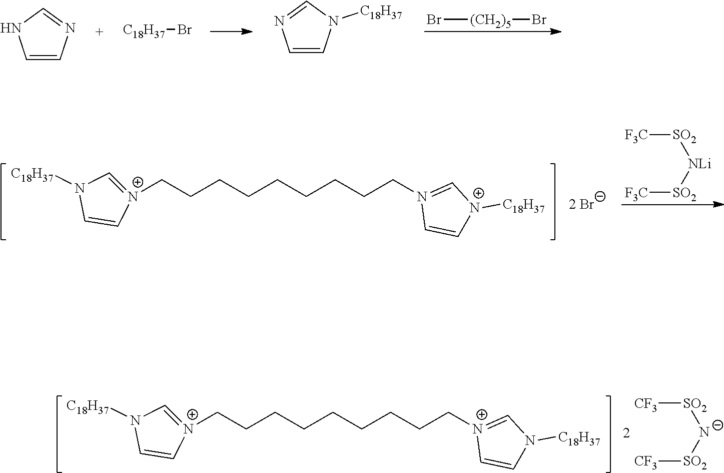

1-Octadecylirnidazole was obtained as follows. Imidazole (3 g) was dissolved in 100 mL of acetonitrile, 1.49 g of octadecyl bromide and 2.51 g of potassium hydroxide were added to the obtained solution, and the resultant was heated to reflux for 4 hours with stirring. After removing the solvent, the generated product was extracted with dichloromethane, followed by refining the product through column chromatography. The resultant was analyzed by gas chromatography. It was found that the product of 1-octadecylimidazole had purity of 98.5% or higher.

In isopropanol, 10.91 g of 1-octadecylimidazole and 4.88 g of 1,9-dibromononane were obtained. The obtained solution was poured into a three-necked flask equipped with THREE-ONE MOTOR and a cooler, and was heated to reflux for 3 hours. After removing the solvent, recrystallization was performed using a mixed solvent of n-hexane and ethanol to thereby obtain 14.11 g of colorless crystals of 1,9-bis(1-octadecylimidazoliumbromide)nonane. The yield was 89%.

In ethanol, 4.54 g of 1,9-bis(1-octadecylimidazoliumbromide)nonane and 2.82 g of lithium trifluoromethanesulfonylimide were dissolved. After stirring the solution for 1 hour at room temperature, the solution was heated to reflux for 2 hours. After completing the reaction, the solvent was removed, and the reaction product was sufficiently washed with water. After vacuum drying the product at 60° C., recrystallization was performed using a mixed solvent of n-hexane and ethanol, to thereby obtain 5.74 g of 1,9-bis[1-octadecylimidazolium bis(trifluoromethanesulfonyl)imide]nonane. The yield was 88%.

In the present specification, the measurement of FTIR was performed by means of FT/IR-460 available from JASCO Corporation according to a transmission method using KBr plates or KBr pellets. The resolution of the measurement was 4 cm⁻¹.

The ¹H-NMR and ¹³C-NMR spectra were measured by means of Varian MercuryPlus 300 nuclear magnetic resonance spectrometer (available from Varian, Inc.). A chemical shift of ¹H-NMR was represented with a unit of ppm as a comparison with an internal standard (a peak of TMS or a deuterated solvent at 0 ppm). Splitting patterns were described by denoting a singlet as s, a doublet as d, a triplet as t, a multiplet as m, and a broad peak as br.

The FTIR absorption of the generated product and the assignment are presented below.

Asymmetric stretching vibrations of SNS were observed at 1,059 cm⁻¹, symmetric stretching vibrations of SO₂ were observed at 1,136 cm⁻¹, symmetric stretching vibrations of CF₃ were observed at 1,184 cm⁻¹, asymmetric stretching vibrations of a SO₂ bond were observed at 1,350 cm⁻¹, bending vibrations of CH₂ were observed at 1,464 cm⁻¹, stretching vibrations unique to an imidazole ring were observed at 1,558 cm⁻¹, symmetric stretching vibrations of CH₂ were observed at 2,848 cm⁻¹, and asymmetric stretching vibrations of CH₂ were observed at 2,916 cm⁻¹.

Moreover, peaks of a proton (¹H)NMR and a carbon (¹³C)NMR in deuterated DMSO are presented below.

¹H-NMR (deuterated DMSO, δ ppm); 0.836 (t, 6H, J=7.2 Hz), 1.150-1.300 (m, 70H), 1.744-1.790 (m, 8H), 4.141 (t, 8H, J=7.2 Hz), 7.783 (d, 2H, J=3 Hz), 7.786 (d, 2H, J=3 Hz), 9.206 (s, 2H)

¹³C-NMR (deuterated DMSO, δ ppm); 14.073, 22.270, 25.627, 25.658, 28.482, 28.894, 29.016, 29.092, 29.230, 29.443, 29.474, 31.473, 49.010, 119.663, 122.624, 136.086

The generated product was determined as 1,9-bis[1-octadecylimidazolium-bis(trifluoromethanesulfonyl)imide]nonane from the spectra above.

Note that, in 1,9-bis[1-octadecylimidazolium-bis(trifluoromethanesulfonyl)imide]nonane, pKa of an acid that is a base of a conjugate base [bis(trifluoromethanesulfonyl)imide] in acetonitrile is 0.3.

### Example 2

### Synthesis of 1,9-bis[1-octadecylimidazoliumhexafluorocyclopropane-1,3-bis(sulfonyl)imide]nonane

Synthesis of 1,9-bis[1-octadecylimidazoliumhexafluorocyclopropane-1,3-bis(sulfonyl)imide]nonane was performed according to the following scheme.

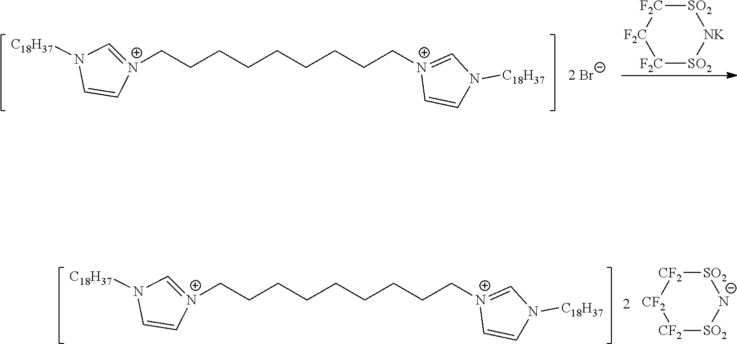

In ethanol, 4.63 g of 1,9-bis(1-octadecylimidazoliumbromide)nonane synthesized in the same manner as in Example 1 and 3.21 g of potassium hexafluorocyclopropane-1,3-bis(sulfonyl)imide were dissolved. After stirring the solution for 1 hour at room temperature, the solution was heated to reflux for 2 hours. After completing the reaction, water was added to the reaction solution to cool. After subjecting to the resultant to filtration, the collected crystals were sufficiently washed with water. After vacuum drying the resultant crystals at 60° C., recrystallization was performed with a mixed solvent of n-hexane and ethanol, to obtain 6.19 g of 1,9-bis[1-octadecylimidazoliumhexafluorocyclopropane-1,3-bis(sulfonyl)imide]nonane. The yield was 93%.

The FTIR absorption of the generated product and the assignment are presented below.

Symmetric stretching vibrations of SO₂ were observed at 1,092 cm⁻¹, symmetric stretching vibrations of CF₂ were observed at 1,159 cm⁻¹, asymmetric stretching vibrations of a SO₂ bond were observed at 1,354 cm⁻¹, bending vibrations of CH₂ were observed at 1,468 cm⁻¹, stretching vibrations unique to imidazole were observed at 1,556 cm⁻¹, symmetric stretching vibrations of CH₂ were observed at 2,848 cm⁻¹, and asymmetric stretching vibrations of CH₂ were observed at 2,916 cm⁻¹.

Moreover, peaks of a proton (¹H)NMR and a carbon (¹³C)NMR in deuterated DMSO are presented below.

¹H-NMR (deuterated DMSO, δ ppm); 0.838 (t, 6H, J=7.2 Hz), 1.170-1.310 (m, 70H), 1.760-1.820 (m, 8H), 4.134 (t, 8H, J=7.2 Hz), 7.773 (d, 2H, J=1.8 Hz), 7.779 (d, 2H, J=1.8 Hz), 9.162 (s, 2H)

¹³C-NMR (deuterated DMSO, δ ppm); 14.362, 22.543, 25.901, 25.962, 28.770, 29.152, 29.274, 29.350, 29.488, 29.701, 29.747, 31.746, 49.299, 122.897, 136.344

The generated product was determined as 1,9-bis[1-octadecylimidazoliumhexafluorocyclopropane-1,3-bis(sulfonyl)imide]nonane from the spectra above.

Note that, in 1,9-bis[1-octadecylimidazoliumhexafluoropropane-1,3-bis(sulfonyl)imide]nonane, pKa of an acid that is a base of a conjugate base [hexafluorocyclopropane-1,3-bis(sulfonyl)imide] in acetonitrile is −0.8.

### Example 3

### Synthesis of 1,9-bis(1-octadecylimidazoliumtricyanomethanide)nonane

Synthesis of 1,9-bis(1-octadecylimidazoliumtricyanomethanide)nonane was performed according to the following scheme.

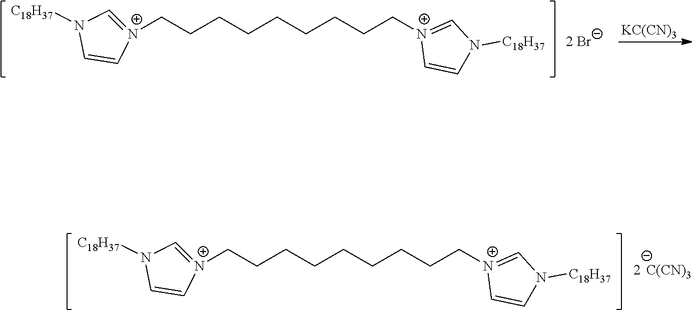

To an ethanol solution including 5.54 g of 1,9-bis(1-octadecylimidazoliumbromide)nonane synthesized in the same manner as in Example 1, a solution prepared by dissolving 1.54 g of potassium tricyanomethanide in water was added. After stirring the solution for 2 hours at room temperature, the solution was heated to reflux for 2 hours. After completing the reaction, the solvent was removed, and the reaction product was sufficiently washed with water. Subsequently, the reaction product was dissolved in a small amount of ethanol, and n-hexane was added to the solution to cool. After collecting the resultant crystals through filtration, the crystals were vacuum dried at 60° C. for 10 hours, to obtain 3.95 g of colorless crystals of 1,9-bis(1-octadecylimidazolium tricyanomethanide)nonane. The yield was 70%.

The FTIR absorption of the generated product and the assignment are presented below.

Bending vibrations of CH₂ were observed at 1,469 cm⁻¹, stretching vibrations unique to imidazole were observed at 1,160 cm⁻¹, stretching vibrations of a CN bond were observed at 2,154 cm⁻¹, symmetric stretching vibrations of CH₂ were observed at 2,848 cm⁻¹, and asymmetric stretching vibrations of CH₂ were observed at 2,914 cm⁻¹.

Moreover, peaks of a proton (¹H)NMR in deuterated DMSO and a carbon (¹³C)NMR in deuterated methanol are presented below.

¹H-NMR (deuterated DMSO, δ ppm); 0.893 (t, 6H, J=7.2 Hz), 1.228-1.371 (m, 70H), 1.810-1.980 (m, 8H), 4.204 (t, 8H, J=7.2 Hz), 7.630 (d, 2H, J=1.2 Hz), 7.634 (d, 2H, J=1.2 Hz), 8.979 (s, 2H)

¹³C-NMR (deuterated methanol, δ ppm); 14.475, 23.740, 27.265, 29.997, 30.059, 30.318, 30.486, 30.532, 30.639, 30.791, 31.051, 31.081, 33.065, 50.908, 122.080, 123.789, 137.068

The generated product was determined as 1,9-bis(1-octadecylimidazolium tricyanomethanide)nonane from the spectra above.

Note that, in 1,9-bis(1-octadecylimidazolium tricyanomethanide)nonane, pKa of an acid that is a base of a conjugate base (tricyanomethanide) in acetonitrile is 5.1.

### Example 4

### Synthesis of 1,5-bis[1-octadecylimidazoliumbis(trifluoromethanesulfonyl)imide]pentane

Synthesis of 1,5-bis[1-octadecylimidazoliumbis(trifluoromethanesulfonyl)imide]pentane was performed according to the following scheme.

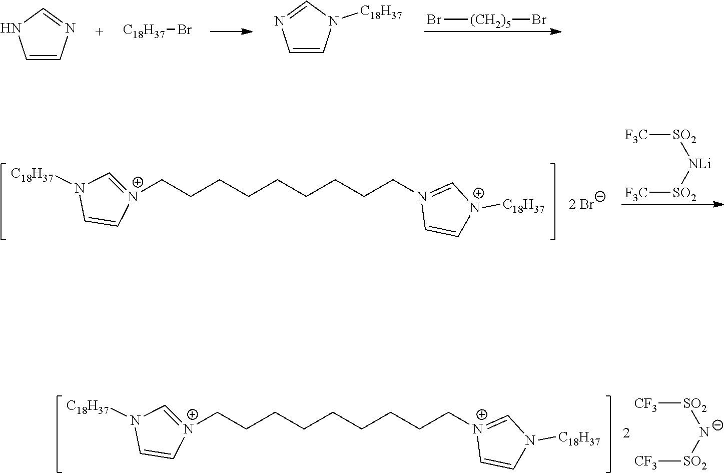

1-Octadecylimidazole was obtained in the following manner. In 100 mL of acetonitrile, 3 g of imidazole was dissolved. To the solution, 14.9 g of octadecyl bromide and 2.51 g of potassium hydroxide were added. The resultant was heated to reflux for 4 hours with stirring. After removing the solvent, the generated product was extracted with dichloromethane, followed by refining the product through column chromatography. As a result of an analysis by gas chromatography, purity of the product of 1-octadecylimidazole was 98.5% or higher.

In isopropanol, 10.00 g of 1-octadecylimidazole and 3.58 g of 1,5-dibromopentane were dissolved. The obtained solution was poured into a three-necked flask equipped with THREE-ONE MOTOR and a cooler, and was heated to reflux for 3 hours. After removing the solvent, recrystallization was performed using a mixed solvent of ethyl acetate and ethanol to thereby obtain 12.82 g of colorless crystals of 1,5-bis(1-octadecylimidazoliumbromide)pentane. The yield was 94%. In water, 3.15 g of 1,5-bis(1-octadecylimidazoliumbromide)pentane was dissolved. To the resultant solution, an aqueous solution including 2.08 g of lithium trifluoromethane sulfonyl imide was added. After stirring the resultant for 1 hour at room temperature, the resultant was heated to reflux for 2 hours. After completing the reaction, crystals were collected through filtration. The collected crystals were sufficiently washed with water until the AgNO₃ test of the filtrate became negative. The resultant was subjected to recrystallization using a mixed solvent of n-hexane and ethanol, to thereby obtain 2.45 g of 1,5-bis[1-octadecylimidazolium bis(trifluoromethanesulfonyl)imide]pentane. The yield was 53.5%.

The FTIR absorption of the generated product and the assignment are presented below.

Asymmetric stretching vibrations of SNS were observed at 1,057 cm⁻¹, symmetric stretching vibrations of SO₂ were observed at 1,134 cm⁻¹, symmetric stretching vibrations of CF₃ were observed at 1,232 cm⁻¹, asymmetric stretching vibrations of a SO₂ bond were observed at 1,352 cm⁻¹, bending vibrations of CH₂ were observed at 1,464 cm⁻¹, symmetric stretching vibrations of CH₂ were observed at 2,850 cm⁻¹, and asymmetric stretching vibrations of CH₂ were observed at 2,918 cm⁻¹.

Moreover, peaks of a proton (¹H)NMR and a carbon (¹³C)NMR in deuterated DMSO are presented below.

¹H-NMR (deuterated DMSO, δ ppm); 0.840 (t, 6H, J=7.2 Hz), 1.120-1.350 (m, 62H), 1.710-1.880 (m, 8H), 4.110-4.169 (m, 8H), 7.756-7.767 (m, 2H), 7.783-7.794 (m, 2H), 9.145 (s, 2H)

¹³C-NMR (deuterated DMSO, δ ppm); 14.089, 22.270, 25.689, 28.543, 28.772, 28.894, 29.016, 29.138, 29.230, 29.504, 31.473, 48.675, 49.041, 119.656 (J=319 Hz), 122.309, 122.670, 136.071

The generated product was determined as 1,5-bis[1-octadecylimidazolium-bis(trifluoromethanesulfonyl)imide]pentane from the spectra above.

Note that, in 1,5-bis[1-octadecylimidazolium-bis(trifluoromethanesulfonyl)imide]pentane, pKa of an acid that is a base of a conjugate base [bis(trifluoromethanesulfonyl)imide] in acetonitrile is 0.3.

### Example 5

### Synthesis of 1,5-bis[1-octadecylimidazolium hexafluorocyclopropane-1,3-bis(sulfonyl)imide]pentane

Synthesis of 1,5-bis[1-octadecylimidazolium hexafluorocyclopropane-1,3-bis(sulfonyl)imide]pentane was performed according to the following scheme.

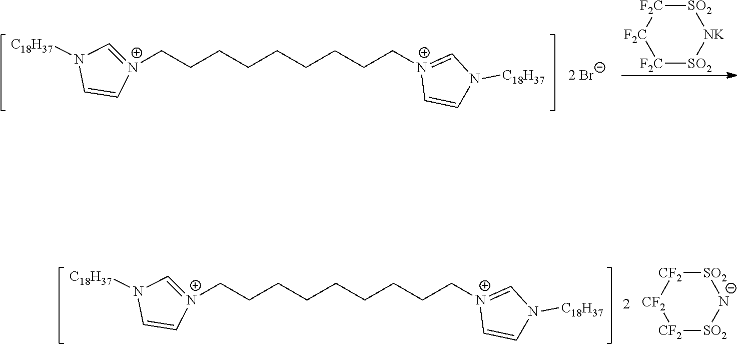

In water, 3.65 g of 1,5-bis(1-octadecylimidazoliumbromide)pentane synthesized in the same manner as in Example 4 was dissolved. To the solution, an aqueous solution including 2.78 g of potassium hexafluorocyclopropane-1,3-bis(sulfonyl)imide was added. After stirring the resultant for 1 hour at room temperature, the resultant was heated to reflux for 2 hours. After completing the reaction, the resultant was cooled, followed by filtration. The generated product was sufficiently washed until the AgNO₃ test of the filtrate became negative. After vacuum drying the product at 60° C., recrystallization was performed using a mixed solvent of n-hexane and ethanol, to thereby obtain 5.07 g of 1,5-bis[1-octadecylimidazolium hexafluorocyclopropane-1,3-bis(sulfonyl)imide]pentane. The yield was 94.9%.

The FTIR absorption of the generated product and the assignment are presented below.

Stretching vibrations of SNS were observed at 1,043 cm⁻¹, symmetric stretching vibrations of SO₂ were observed at 1,090 cm⁻¹, symmetric stretching vibrations of CF₂ were observed at 1,155 cm⁻¹, asymmetric stretching vibrations of a SO₂ bond were observed at 1,350 cm⁻¹, bending vibrations of CH₂ were observed at 1,469 cm⁻¹, symmetric stretching vibrations of CH₂ were observed at 2,850 cm⁻¹, and asymmetric stretching vibrations of CH₂ were observed at 2,920 cm⁻¹.

Moreover, peaks of a proton (¹H)NMR and a carbon (¹³C)NMR in deuterated DMSO are presented below.

¹H-NMR (deuterated DMSO, δ ppm); 0.839 (t, 6H, J=7.2 Hz), 1.170-1.310 (m, 62H), 1.710-1.870 (m, 8H), 4.109-4.168 (m, 8H), 7.753-7.765 (m, 2H), 7.781-7.792 (m, 2H), 9.141 (s, 2H)

¹³C-NMR (deuterated DMSO, δ ppm); 14.089, 22.270, 25.689, 28.543, 28.787, 28.894, 29.016, 29.138, 29.230, 29.504, 31.473, 48.675, 49.056, 122.609, 122.655, 136.071

The generated product was determined as 1,5-bis[1-octadecylimidazolium hexafluorocyclopropane-1,3-bis(sulfonyl)imide]pentane from the spectra above.

Note that, in 1,5-bis[1-octadecylimidazolium hexafluorocyclopropane-1,3-bis(sulfonyl)imide]pentane, pKa of an acid that is a base of a conjugate base [hexafluorocyclopropane-1,3-bis(sulfonyl)imide] in acetonitrile was −0.8.

### Example 6

### Synthesis of 1,5-bis[1-octadecylimidazolium-bis(nonafluorobutanesulfonyl)imide]pentane

Synthesis of 1,5-bis[1-octadecylimidazolium-bis(nonafluorobutanesulfonyl)imide]pentane was performed according to the following scheme.

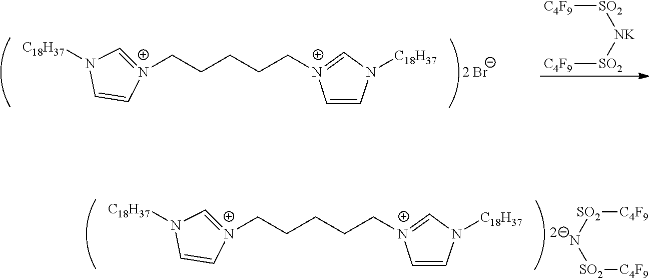

In water, 1:93 g of 1,5-bis(1-octadecylimidazoliumbromide)pentane synthesized in the same manner as in Example 4 was dissolved. To the solution, 2.76 g of potassium (nonafluorobutanesulfonyl)imide dissolved in a small amount of ethanol was added. After stirring the resultant for 2 hours at room temperature, the resultant was heated to reflux for 2 hours. After completing the reaction, filtration was performed, and the collected product was sufficiently washed with water until the AgNO₃ test of the filtrate became negative. The resultant was vacuum dried at 70° C. for 10 hours, to thereby obtain 4.00 g of a waxy compound of 1,5-bis[1-octadecylimidazolium-bis(nonafluorobutanesulfonyl)imide]pentane. The yield was 96.5%.

The FTIR absorption of the generated product and the assignment are presented below.

Symmetric stretching vibrations of SO₂ were observed at 1,072 cm⁻¹, symmetric stretching vibrations of CF₂ were observed at 1,167 cm⁻¹, asymmetric stretching vibrations of a SO₂ bond were observed at 1,352 cm⁻¹, bending vibrations of CH₂ were observed at 1,468 cm⁻¹, stretching vibrations of C═N were observed at 1,566 cm⁻¹, symmetric stretching vibrations of CH₂ were observed at 2,854 cm⁻¹, and asymmetric stretching vibrations of CH₂ were observed at 2,924 cm⁻¹.

Moreover, peaks of a proton (¹H)NMR and a carbon (¹³C)NMR in deuterated DMSO are presented below.

¹H-NMR (deuterated DMSO, δ ppm); 0.828 (t, 6H, J=6.9 Hz), 1.130-1.340 (m, 62H), 1.710-1.880 (m, 8H), 4.106-4.169 (m, 8H), 7.750-7.761 (m, 2H), 7.772-7.782 (m, 2H), 9.141 (s, 2H)

¹³C-NMR (deuterated DMSO, δ ppm); 13.967, 22.239, 25.689, 28.543, 28.772, 28.879, 29.016, 29.138, 29.214, 29.520, 31.458, 48.675, 49.041, 122.594, 122.639, 136.086

The generated product was determined as 1,5-bis[1-octadecylimidazolium-bis(nonafluorobutanesulfonyl)imide]pentane from the spectra above.

Note that, in 1,5-bis[1-octadecylimidazolium-bis(nonafluorobutanesulfonyl)imide]pentane, pKa of an acid that is a base of a conjugate base [bis(nonafluorobutanesulfonyl)imide] in acetonitrile is 0.0.

### Example 7

### Synthesis of 1,9-bis[1-octadecylpyrrolidinium bis(trifluoromethanesulfonyl)imide]nonane

Synthesis of 1,9-bis[1-octadecylpyrrolidinium bis(trifluoromethanesulfonyl)imide]nonane was performed according to the following scheme.

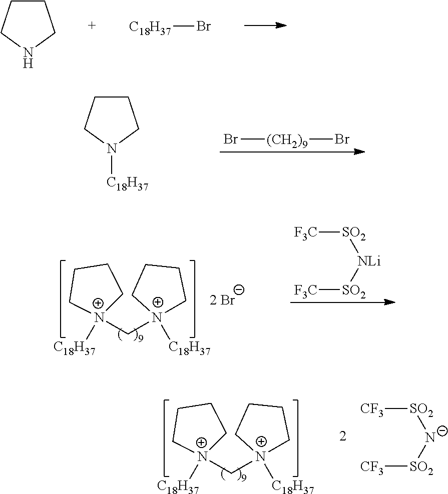

1-Octadecylpyrrolidine was obtained in the following manner. In 100 mL of acetonitrile, 5.93 g of pyrrolidine was dissolved. To the solution, 27.74 g of octadecyl bromide and 4.67 g of potassium hydroxide were added. The resultant was heated to reflux with stirring for 12 hours. After removing the solvent, the generated product was extracted with dichloromethane, and the extracted product was refined by column chromatography using a solution including n-hexane and ethyl acetate at the ratio of 5:1 as a solvent. The resultant was analyzed by gas chromatography. As a result, the product of 1-octadecylpyrrolidine had purity of 98.5% or higher.

In isopropanol, 6.87 g of 1-octadecylpyrrolidine and 3.04 g of 1,9-dibromononane were dissolved. The obtained solution was poured into a three-necked flask equipped with THREE-ONE MOTOR and a cooler, and was heated to reflux for 32 hours. After removing the solvent, the resultant was dissolved in water, followed by washing 3 times with 20 mL of ethyl acetate. After drying the aqueous phase, the resultant was further washed with ethyl acetate, to thereby obtain 7.00 g of colorless crystals of 1,9-bis(bromo-1-octadecylpyrrolidinium)nonane. The yield was 71%.

In water, 3.00 g of 1,9-bis(bromo-1-octadecylpyrrolidinium)nonane was dissolved. To the solution, an aqueous solution including 1.86 g of lithium trifluoromethane sulfoimide was added. After stirring the resultant for 1 hour at room temperature, the resultant was heated to reflux for 1 hour. After completing the reaction, crystals were collected through filtration. After sufficiently washing the collected crystals with water until the AgNO₃ test of the filtrate became negative, the resultant was dried. The resultant was subjected to recrystallization using a mixed solvent of n-hexane and ethanol to thereby obtain 2.46 g of 1,9-bis[1-octadecylpyrrolidinium bis(trifluoromethanesulfonyl)imide]nonane. The yield was 57%.

The FTIR absorption of the generated product and the assignment are presented below.

Asymmetric stretching vibrations of SNS were observed at 1,074 cm⁻¹, symmetric stretching vibrations of SO₂ were observed at 1,134 cm⁻¹, symmetric stretching vibrations of CF₃ were observed at 1,250 cm⁻¹, bending vibrations of CH₂ were observed at 1,468 cm⁻¹, symmetric stretching vibrations of CH₂ were observed at 2,848 cm⁻¹, and asymmetric stretching vibrations of CH₂ were observed at 2,916 cm⁻¹.

Moreover, peaks of a proton (¹H)NMR and a carbon (¹³C)NMR in deuterated DMSO are presented below.

¹H-NMR (deuterated DMSO, δ ppm); 0.843 (t, 6H, J=6.9 Hz), 1.170-1.360 (m, 70H), 1.530-1.650 (m, 8H), 2.000-2.060 (m, 8H), 3.120-3.210 (m, 8H), 3.390-3.480)

¹³C-NMR (deuterated DMSO, δ ppm); 14.058, 21.537, 22.270, 22.560, 22.651, 25.963, 26.024, 28.619, 28.650, 28.909, 28.985, 29.123, 29.245, 31.488, 58.794, 62.274, 119.663 (J=320 Hz),

The generated product was determined as 1,9-bis[1-octadecylpyrrolidinium bis(trifluoromethanesulfonyl)imide]nonane from the spectra above.

Note that, in 1,9-bis[1-octadecylpyrrolidinium bis(trifluoromethanesulfonyl)imide]nonane, pKa of an acid that is a base of a conjugate base [bis(trifluoromethanesulfonyl)imide] in acetonitrile is 0.3.

### Example 8

### Synthesis of 1,9-bis[1-octadecylpyrrolidinium hexafluorocyclopropane-1,3-bis(sulfonyl)imide]nonane

Synthesis of 1,9-bis[1-octadecylpyrrolidinium hexafluorocyclopropane-1,3-bis(sulfonyl)imide]nonane was performed according to the following scheme.

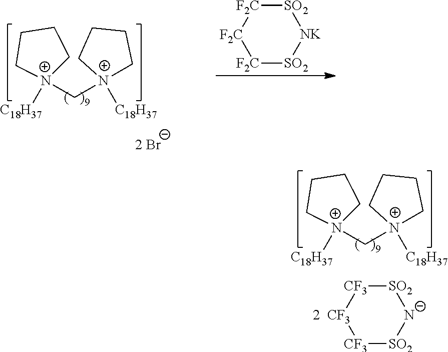

In water, 3.56 g of 1,9-bis(bromo-N-octadecylpyrrolidinium)nonane in the same manner as in Example 7 was dissolved. To the solution, an aqueous solution including 2.44 g of potassium hexafluorocyclopropane-1,3-bis(sulfonyl)imide was added. After stirring the resultant for 1 hour at room temperature, the resultant was heated to reflux for 1 hour. After completing the reaction, crystals were collected through filtration. The collected crystals were sufficiently washed with water until the AgNO₃ test of the filtrate became negative, followed by drying. The resultant was subjected to recrystallization using ethanol, to thereby obtain 3.53 g of 1,9-bis[1-octadecylpyrrolidinium hexafluorocyclopropane-1,3-bis(sulfonyl)imide]nonane. The yield was 69%.

The FTIR absorption of the generated product and the assignment are presented below.

Symmetric stretching vibrations of CF₂ were observed at 1,155 cm⁻¹, symmetric stretching vibrations of SO₂ were observed at 1,350 cm⁻¹, bending vibrations of CH₂ were observed at 1,468 cm⁻¹, symmetric stretching vibrations of CH₂ were observed at 2,850 cm⁻¹, and asymmetric stretching vibrations of CH₂ were observed at 2,920 cm⁻¹.

Moreover, peaks of a proton (¹H)NMR and a carbon (¹³C)NMR in deuterated DMSO are presented below.

¹H-NMR (deuterated DMSO, δ ppm); 0.842 (t, 6H, J=6.9 Hz), 1.170-1.330 (m, 70H), 1.530-1.650 (m, 8H), 2.000-2.060 (m, 8H), 3.120-3.210 (m, 8H), 3.390-3.480)

¹³C-NMR (deuterated DMSO, δ ppm); 14.119, 21.537, 22.270, 22.544, 22.666, 25.948, 26.040, 28.619, 28.695, 28.894, 28.970, 29.107, 29.212, 31.473, 58.748, 62.274

The generated product was determined as 1,9-bis[1-octadecylpyrrolidiniumhexafluorocyclopropane-1,3-bis(sulfonyl)imide]nonane from the spectra above.

Note that, in 1,9-bis[1-octadecylpyrrolidiniumhexafluorocyclopropane-1,3-bis(sulfonyl)imide]nonane, pKa of an acid that is a base of a conjugate base, hexafluorocyclopropane-1,3-bis(sulfonyl)imide, in acetonitrile is −0.8.

### Example 9

### Synthesis of 1,9-bis[6-octadecyl-1,8-diazabicyclo[5.4.0]-7-undeceniumhexafluorocyclopropane-1,3-bis (sulfonyl)imide]nonane

Synthesis of 1,9-bis[6-octadecyl-1,8-diazabicyclo[5.4.0]-7-undeceniumhexafluorocyclopropane-1,3-bis (sulfonyl)imide]nonane was performed according to the following scheme.

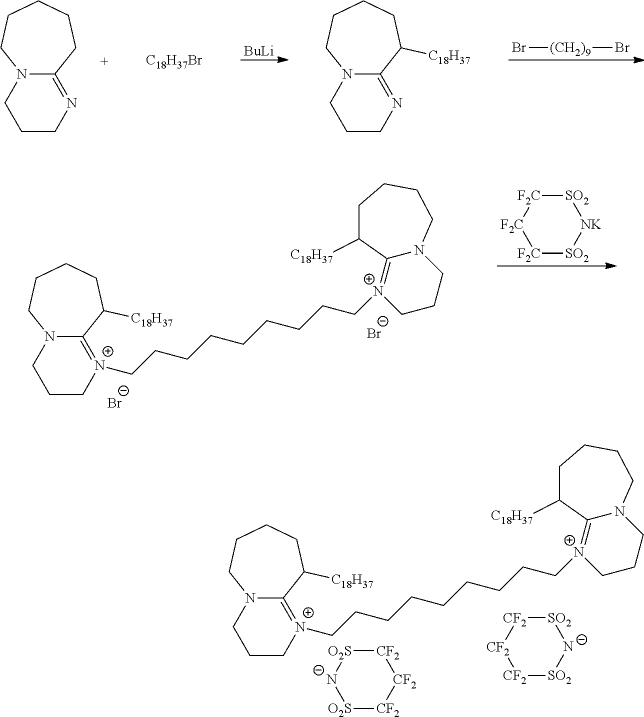

6-Octadecyl-1,8-diazabicyclo[5.4.0]-7-undecene was synthesized by the method of Murayama et al. (NPL: N. Matsumura, H. Nishiguchi, M. Okada, and S. Yoneda, J. Heterocyclic Chem. pp. 885-887, Vol. 23, Issue 3 (1986)). Specifically, 1,8-diazabicyclo[5.4.0]-7-undecene (DBU) was dissolved in tetrahydrofuran. When the resultant solution was cooled to 0° C., butyl lithium was added to the solution by dripping, and the resultant solution was stirred for 1 hour at 0° C. To the solution, a THF solution of octadecyl bromide that was equimolar to DBU was added by dripping. After completing the reaction, the reaction solution was adjusted to be acidic with dilute hydrochloric acid, and the generated product was extracted with diethyl ether. The aqueous solution was adjusted to be alkaline with sodium hydroxide, followed by performing extraction with diethyl ether. The diethyl ether phase was washed with water, followed by drying with anhydrous magnesium sulfate to remove the solvent. Thereafter, the resultant was refined by silica-gel chromatography. The yield was 75%.

In isopropanol, the equimolar of 1,9-dibromononane to the obtained 6-octadecyl-1,8-diazabicyclo[5.4.0]-7-undecene was dissolved. The resultant solution was heated to reflux for 48 hours. After completing the reaction, the solvent was removed. The resultant was dissolved in water to extract impurities and unreacted products with ethyl acetate. The solvent was removed from the aqueous phase to thereby obtain a dibromo compound. The yield was 79%.

The obtained dibromo compound was dissolved in water. To the solution, a solution prepared by dissolving potassium hexafluorocyclopropane-1,3-bis(sulfonyl)imide in water and a small amount of ethanol was added. After stirring the resultant for 1 hour, the resultant was heated to reflux for 1 hour. After cooling the resultant, the precipitates were washed with water until the AgNO₃ test became negative, followed by vacuum drying the precipitates for 12 hours at 100° C. The yield was 70%.

The FTIR absorption of the generated product and the assignment are presented below.

Asymmetric stretching vibrations of SNS were observed at 1,036 cm⁻¹, symmetric stretching vibrations of SO₂ were observed at 1,088 cm⁻¹, symmetric stretching vibrations of CF₂ were observed at 1,153 cm⁻¹, asymmetric stretching vibrations of SO₂ were observed at 1,352 cm⁻¹, stretching vibrations of C═N were observed at 1,618 cm⁻¹, symmetric stretching vibrations of CH₂ were observed at 2,850 cm⁻¹, and asymmetric stretching vibrations of CH₂ were observed at 2,920 cm⁻¹.

Moreover, peaks of a proton (¹H)NMR and a carbon (¹³C)NMR in CD₃OD are presented below.

¹H-NMR (CD₃OD, δ ppm); 0.872 (t, 6H, J=6.9 Hz), 1.220-1.460 (m, 74H), 1.631-1.679 (m, 4H), 1.708-1.810 (m, 12H), 2.031-2.109 (in, 4H), 2.854-2.887 (m, 2H), 3.491-3.543 (m, 12H), 3.633-3.663 (m, 4H)

¹³C-NMR (CD₃OD, δ ppm); 14.024, 19.336, 21.038, 22.633, 24.182, 25.121, 26.311, 27.021, 27.181, 27.387, 28.311, 29.005, 29.028, 29.303, 29.425, 29.532, 29.601, 29.608, 29.646, 29.654, 30.211, 30.272, 31.882, 38.491, 50.114, 55.014, 55.914, 167.915

The generated product was determined as 1,9-bis[6-octadecyl-1,8-diazabicyclo[5.4.0]-7-undeceniumhexafluorocyclopropane-1,3-bis (sulfonyl)imide]nonane from the spectra above.

Note that, in 1,9-bis[6-octadecyl-1,8-diazabicyclo[5.4.0]-7-undeceniumhexafluorocyclopropane-1,3-bis (sulfonyl)imide]nonane, pKa of an acid that is a base of a conjugate base [hexafluorocyclopropane-1,3-bis(sulfonyl)imide] in acetonitrile is −0.8.

### Example 10

### Synthesis of 1,3-bis[1-octadecylimidazolium bis(nonafluorobutanesulfonyl)imide]propane

Synthesis of 1,3-bis[1-octadecylimidazolium bis(nonafluorobutanesulfonyl)imide]propane was performed according to the following scheme.

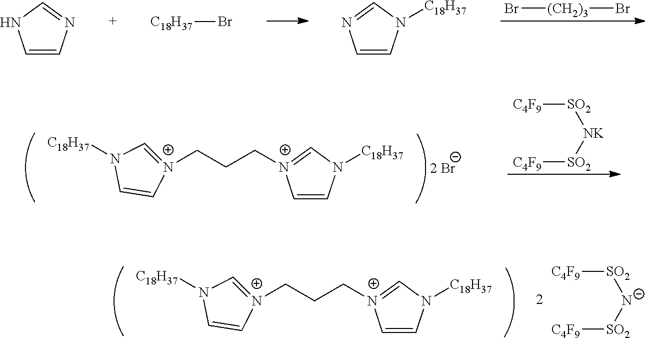

In isopropanol, 11.51 g of 1-octadecylimidazole synthesized in the same manner as in Example 1 and 3.60 g of 1,3-dibromopropane were dissolved. The obtained solution was poured into a three-necked flask equipped with THREE-ONE MOTOR and a cooler, and was heated to reflux for 3 hours. After removing the solvent, the obtained crystals were sufficiently washed with ethyl acetate, followed by performing recrystallization using a mixed solvent of n-hexane and ethanol, to thereby obtain 13.37 g of colorless crystals of 1,3-bis(1-octadecylimidazoliumbromide)propane. The yield was 89%.

In ethanol, 2.49 g of 1,3-bis(1-octadecylimidazoliumbromide)propane and 3.70 g of lithium nonafluorobutane sulfonylimide were dissolved. After stirring the resultant solution for 1 hour at room temperature, the solution was heated to reflux for 2 hours. After completing the reaction, the solvent was removed, and the resultant was dissolved in dichloromethane, followed by sufficiently washing with water. The resultant was subjected to recrystallization using a mixed solvent of n-hexane and ethanol, to thereby obtain 4.80 g of 1,3-bis[1-octadecylimidazolium bis(nonafluorobutanesulfonyl)imide]propane. The yield was 85%.

The FTIR absorption of the generated product and the assignment are presented below.

Asymmetric stretching vibrations of SNS were observed at 1,072 cm⁻¹, symmetric stretching vibrations of CF₂ were observed at 1,167 cm⁻¹, asymmetric stretching vibrations of a SO₂ bond were observed at 1,352 cm⁻¹, out-of-plane bending vibrations of CH₂ were observed at 1,468 cm⁻¹, stretching vibrations of C═N were observed at 1,566 cm⁻¹, symmetric stretching vibrations of CH₂ were observed at 2,854 cm⁻¹, and asymmetric stretching vibrations of CH₂ were observed at 2,924 cm⁻¹.

Moreover, peaks of a proton (¹H)NMR and a carbon (¹³C)NMR in CDCl₃ are presented below.

¹H-NMR (CDCl₃, δ ppm); 0.854 (t, 6H, J=7.2 Hz), 1.150-1.350 (m, 60H), 1.780-1.900 (m, 4H), 2.481-2.587 (m, 2H), 4.098 (t, 41-1, J=7.2 Hz), 4.359 (t, 41-1, J=7.2 Hz), 7.189-7.200 (m, 2H), 7.600-6.610 (m, 2H), 9.206 (s, 2H), 8.333 (2H, s)

¹³C-NMR (CDCl₃, δ ppm); 14.085, 22.679, 26.143, 28.799, 29.257, 29.349, 29.455, 29.547, 29.654, 29.684, 29.883, 31.470, 31.913, 46.413, 50.412, 122.057, 123.308, 135.458

The generated product was determined as 1,3-bis[1-octadecylimidazolium-bis(nonafluorobutanesulfonyl)imide]propane from the spectra above.

Note that, in 1,3-bis[1-octadecylimidazolium-bis(nonafluorobutanesulfonyl)imide]propane, pKa of an acid that is a base of a conjugate base [bis(nonafluorobutanesulfonyl)imide] is 0.0.

### Example 11

### Synthesis of 1,3-bis[1-octadecylimidazolium hexafluorocyclopropane-1,3-bis(sulfonyl)imide]propane

Synthesis of 1,3-bis[1-octadecylimidazolium hexafluorocyclopropane-1,3-bis(sulfonyl)imide]propane was performed according to the following scheme.

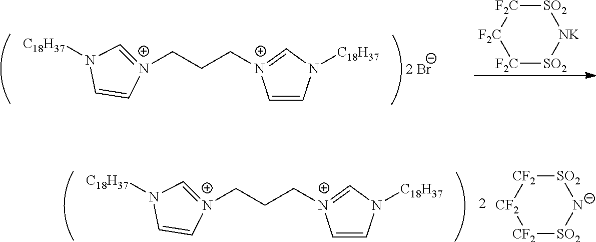

In an ethanol aqueous solution, 2.78 g of 1,3-bis(1-octadecylimidazoliumbromide)propane synthesized in the same manner as in Example 10 and 2.40 g of potassium hexafluorocyclopropane-1,3-bis(sulfonyl)imide were dissolved. After stirring the resultant solution for 1 hour at room temperature, the solution was heated to reflux for 2 hours. After completing the reaction, water was added to the reaction solution to cool, followed by performing filtration. The collected crystals were sufficiently washed with water. After vacuum drying the crystals at 60° C., recrystallization was performed using a mixed solvent of n-hexane and ethanol, to thereby obtain 3.62 g of 1,3-bis[1-octadecylimidazolium hexafluorocyclopropane-1,3-bis(sulfonyl)imide]propane. The yield was 91%.

The FTIR absorption of the generated product and the assignment are presented below.

Symmetric stretching vibrations of SO₂ were observed at 1,092 cm⁻¹, symmetric stretching vibrations of CF₂ were observed at 1,159 cm⁻¹, asymmetric stretching vibrations of a SO₂ bond were observed at 1,354 cm⁻¹, out-of-plane bending vibrations of CH₂ were observed at 1,468 cm⁻¹, stretching vibrations of C═N were observed at 1,556 cm⁻¹, symmetric stretching vibrations of CH₂ were observed at 2,848 cm⁻¹, and asymmetric stretching vibrations of CH₂ were observed at 2,916 cm⁻¹.

Moreover, peaks of a proton (¹H)NMR and a carbon (¹³C)NMR in CDCl₃ are presented below.

¹H-NMR (CDCl₃, δ ppm); 0.854 (t, 6H, J=7.2 Hz), 1.140-1.360 (m, 60H), 1.770-1.900 (m, 4H), 2.470-2.600 (m, 2H), 4.112 (t, 4H, J=7.2 Hz), 4.356 (t, 4H, J=7.2 Hz), 7.192-7.204 (m, 2H), 7.570-7.582 (m, 2H), 8.768 (s, 2H)

¹³C-NMR (CDCl₃, δ ppm); 14.085, 22.663, 26.174, 28.845, 29.272, 29.349, 29.471, 29.578, 29.639, 29.684, 29.898, 31.333, 31.913, 46.550, 50.381, 122.118, 123.186, 135.503

The generated product was determined as 1,3-bis[1-octadecylimidazolium hexafluorocyclopropane-1,3-bis(sulfonyl)imide]propane from the spectra above.

Note that, in 1,3-bis[1-octadecylimidazolium hexafluorocyclopropane-1,3-bis(sulfonyl)imide]propane, pKa of an acid that is a base of a conjugate base [hexafluorocyclopropane-1,3-bis(sulfonyl)imide] in acetonitrile is −0.8.

### Example 12

### Synthesis of 1,9-bis[1-octadecylimidazolium bis(nonafluorobutanesulfonyl)imide]nonane

Synthesis of 1,9-bis[1-octadecylimidazolium bis(trifluoromethanesulfonyl)imide]nonane was performed according to the following scheme.

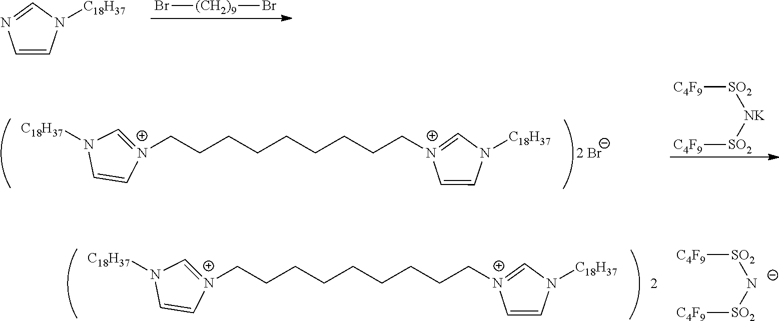

In water, 3.00 g of 1,9-bis(1-octadecylimidazoliumbromide)nonane synthesized in the same manner as in Example 1 was dissolved. To the resultant solution, an aqueous solution including 4.04 g of lithium bis(nonafluorobutanesulfonyl)imide was added. After stirring the resultant for 1 hour at room temperature, the resultant was heated to reflux for 2 hours. After completing the reaction, crystals were collected through filtration. The crystals were sufficiently washed with water until the AgNO₃ test of the filtrate became negative, to thereby obtain 5.16 g of 1,9-bis[1-octadecylimidazolium bis(nonafluorobutanesulfonyl)imide]pentane. The yield was 73.7%.

The FTIR absorption of the generated product and the assignment are presented below.

Asymmetric stretching vibrations of SNS were observed at 1,072 cm⁻¹, symmetric stretching vibrations of CF₂ were observed at 1,169 cm⁻¹, asymmetric stretching vibrations of a SO₂ bond were observed at 1,352 cm⁻¹, bending vibrations of CH₂ were observed at 1,469 cm⁻¹, symmetric stretching vibrations of C═N were observed at 1,564 cm⁻¹, symmetric stretching vibrations of CH₂ were observed at 2,850 cm⁻¹, and asymmetric stretching vibrations of CH₂ were observed at 2,920 cm⁻¹.

Moreover, peaks of a proton (¹H)NMR and a carbon (¹³C)NMR in CDCl₃ are presented below.

¹H-NMR (CDCl₃, δ ppm); 0.870 (t, 6H, J=6.9 Hz), 1.180-1.370 (m, 70H), 1.780-1.920 (m, 8H), 4.151 (q, 8H, J=7.2 Hz), 7.244-7.257 (m, 2H), 7.398-7.410 (m, 2H), 8.816 (s, 2H)

¹³C-NMR (CDCl₃, δ ppm); 14.055, 22.663, 25.334, 26.082, 27.807, 27.929, 28.845, 29.288, 29.333, 29.455, 29.562, 29.669, 29.730, 30.127, 31.898, 50.015, 50.091, 121.980, 122.621, 135.366

The generated product was determined as 1,9-bis[1-octadecylimidazolium-bis(nonafluoromethanesulfonyl)imide]nonane from the spectra above.

Note that, in 1,9-bis[1-octadecylimidazolium-bis(nonafluoromethanesulfonyl)imide]nonane, pKa of an acid that is a base of a conjugate base [bis(nonafluoromethanesulfonyl)imide] in acetonitrile is 0.0.

### Example 13

### Synthesis of 1,9-bis[1-octadecylimidazoliumnonafluorobutanesulfonium]nonane

Synthesis of 1,9-bis[1-octadecylimidazoliumnonafluorobutanesulfonium]nonane was performed according to the following scheme.

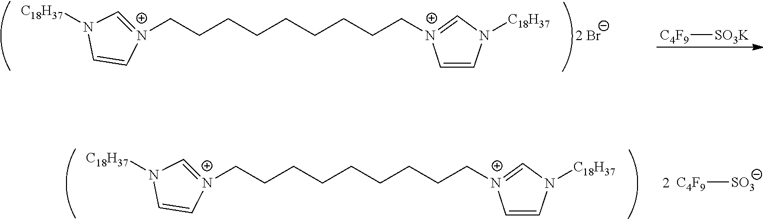

In water, 2.48 g of 1,9-bis(1-octadecylimidazoliumbromide)nonane synthesized in the same manner as in Example 12 was dissolved. To the solution, an aqueous solution including 1.93 g of potassium nonafluorobutane sulfonate was added. After stirring the resultant for 1 hour at room temperature, the resultant was heated to reflux for 2 hours. After completing the reaction, the reaction liquid was cooled, followed by filtration. The resultant was sufficiently washed with water until the AgNO₃ test of the filtrate became negative. After vacuum drying the reaction product at 60° C., recrystallization was performed using a mixed solvent of n-hexane and ethanol, to thereby obtain 3.03 g of 1,9-bis[1-octadecylimidazoliumnonafluorobutanesulfonium]nonane. The yield was 83.0%.

The FTIR absorption of the generated product and the assignment are presented below.

Symmetric stretching vibrations of SO₂ were observed at 1,134 cm⁻¹, symmetric stretching vibrations of CF were observed at 1,255 cm⁻¹, asymmetric stretching vibrations of a SO₂ bond were observed at 1,352 cm⁻¹, bending vibrations of CH₂ were observed at 1,468 cm⁻¹, symmetric stretching vibrations of CH₂ were observed at 2,850 cm⁻¹, and asymmetric stretching vibrations of CH₂ were observed at 2,918 cm⁻¹.

Moreover, peaks of a proton (¹H)NMR and a carbon (¹³C)NMR in deuterated chloroform are presented below.

¹H-NMR (CDCl₃, δ ppm); 0.850 (t, 6H, J=6.9 Hz), 1.173-1.350 (m, 70H), 1.770-1920 (m, 8H), 4.180 (q, 81-1, J=7.2 Hz), 7.270-7.282 (m, 2H), 7.471-7.482 (m, 2H), 9.214 (s, 2H)

¹³C-NMR (CDCl₃, δ ppm); 14.085, 22.663, 25.258, 26.159, 27.654, 27.914, 28.906, 29.333, 29.471, 29.578, 29.639, 29.684, 30.173, 31.898, 49.893, 50.076, 122.904, 122.637, 136.190

The generated product was determined as 1,9-bis[1-octadecylimidazoliumnonafluorobutanesulfonium]nonane from the spectra above.

Note that, in 1,9-bis[1-octadecylimidazoliumnonafluorobutanesulfonium]nonane, pKa of an acid that is a base of a conjugate base (nonafluorobutane sulfonic acid) in acetonitrile is 0.7.

### Example 14

### Synthesis of 1,9-bis[dimethyloctadecylammonium-bis(nonafluorobutanesulfonyl)imide]nonane

Synthesis of 1,9-bis[dimethyloctadecylammonium-bis(nonafluorobutanesulfonyl)imide]nonane was performed according to the following scheme.

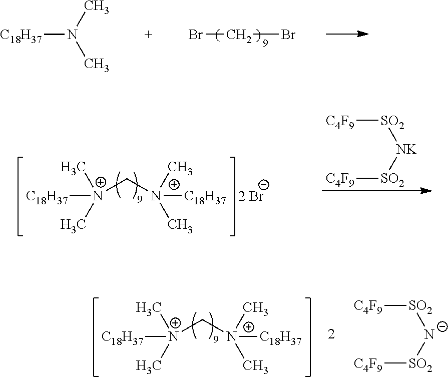

In 2-propanol, 9.89 g of dimethyloctadecyl amine and 4.55 g of 1,9-dibromononane were added, and the resultant was heated to reflux for 3 hours. After cooling the resultant, the solvent was removed, and recrystallization was performed using a mixed solvent of n-hexane and ethanol, to thereby obtain 12.63 g of 1,9-bis[dimethyloctadecylammoniumbromide]nonane. The yield was 90.2%.

In a mixed solvent of water and ethanol, 2.02 g of the obtained 1,9-bis[dimethyloctadecylammoniumbromide]nonane and 2.88 g of bis(nonafluorobutanesulfonyl)imide were dissolved. The resultant solution was heated to reflux. After removing the solvent from the solution, the resultant was sufficiently washed with water until the AgNO₃ test became negative. Thereafter, the resultant was vacuum dried for 5 hours at 60° C., and recrystallization was performed using a mixed solvent of n-hexane and ethanol, to thereby obtain 2.75 g of 1,9-bis[dimethyloctadecylammonium-bis(nonafluorobutanesulfonyl)imide]nonane. The yield was 60.8%.

The FTIR absorption of the generated product and the assignment are presented below.

Asymmetric stretching vibrations of SNS were observed at 1,030 cm⁻¹, symmetric stretching vibrations of SO₂ were observed at 1,080 cm⁻¹, symmetric stretching vibrations of CF were observed at 1,136 cm⁻¹ and 1,192 cm⁻¹, asymmetric stretching vibrations of a SO₂ bond were observed at 1,344 cm⁻¹, bending vibrations of CH₂ were observed at 1,469 cm⁻¹, symmetric stretching vibrations of CH₂ were observed at 2,852 cm⁻¹, and asymmetric stretching vibrations of CH₂ were observed at 2,922 cm⁻¹.

Moreover, peaks of a proton (¹H)NMR and a carbon (¹³C)NMR in deuterated chloroform are presented below.

¹H-NMR (CDCl₃, δ ppm); 0.857 (t, 6H, J=6.6 Hz), 1.160-1.400 (m, 70H), 1.600-1.780 (m, 8H), 2.998 (s, 12H), 3.148-3.246 (m, 4H), 3.262-3.303 (m, 4H)

¹³C-NMR (CDCl₃, δ ppm); 14.085, 22.129, 22.679, 25.075, 26.036, 27.425, 27.670, 29.043, 29.303, 29.349, 29.394, 29.562, 29.623, 29.684, 31.913, 50.396, 65.095

The generated product was determined as 1,9-bis[dimethyloctadecylammonium-bis(nonafluorobutanesulfonyl)imide]nonane from the spectra above.

Note that, in 1,9-bis[dimethyloctadecylammonium-bis(nonafluorobutanesulfonyl)imide]nonane, pKa of an acid that is a base of a conjugate base [bis(nonafluorobutanesulfonyl)imide] in acetonitrile is 0.0.

### Example 15

### Synthesis of 1,9-bis[dimethyloctadecylammonium-hexafluorocyclopropane-1,3-bis(sulfonyl)imide]nonane

Synthesis of 1,9-bis[dimethyloctadecylammonium-hexafluorocyclopropane-1,3-bis(sulfonyl)imide]nonane was performed according to the following scheme.

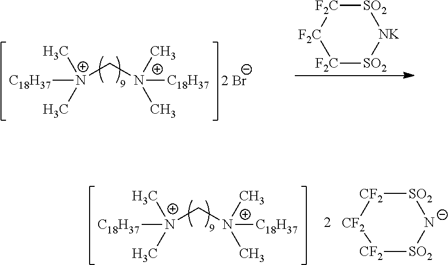

In water, 2.91 g of 1,9-bis[dimethyloctadecylammoniumbromide]nonane synthesized in the same manner as in Example 14 was dissolved. To the solution, an aqueous solution including 1.98 g of lithium hexafluorocyclopropane-1,3-bis(sulfonyl)imide was added. After stirring the resultant for 1 hour at room temperature, the resultant was heated to reflux for 1 hour. After completing the reaction, crystals were collected through filtration. After sufficiently washing the crystals with water until the AgNO₃ test of the filtrate became negative, the crystals were dried, followed by subjected to recrystallization using a mixed solvent of n-hexane and ethanol to thereby obtain 3.61 g of 1,9-bis[dimethyloctadecylammonium-hexafluorocyclopropane-1,3-bis(sulfonyl)imide]nonane. The yield was 83.8%.

The FTIR absorption of the generated product and the assignment are presented below.

Asymmetric stretching vibrations of SNS were observed at 1,039 cm⁻¹, symmetric stretching vibrations of SO₂ were observed at 1,090 cm⁻¹, symmetric stretching vibrations of CF₂ were observed at 1,153 cm⁻¹, asymmetric stretching vibrations of SO₂ were observed at 1,350 cm⁻¹, bending vibrations of CH₂ were observed at 1,469 cm⁻¹ symmetric stretching vibrations of CH₂ were observed at 2,850 cm⁻¹, and asymmetric stretching vibrations of CH₂ were observed at 2,918 cm⁻¹.

Moreover, peaks of a proton (¹H)NMR and a carbon (¹³C)NMR in deuterated chloroform are presented below.

¹H-NMR (CDCl₃, δ ppm); 0.855 (t, 6H, J=6.6 Hz), 1.150-1.430 (m, 70H), 1.600-1.790 (m, 8H), 2.998 (s, 12H), 3.147-3.203 (m, 4H), 3.235-3.292 (m, 4H)

¹³C-NMR (CDCl₃, δ ppm); 14.085, 22.221, 22.618, 22.663, 25.365, 26.052, 27.868, 28.036, 28.998, 29.318, 29.349, 29.425, 29.578, 29.654, 29.700, 31.913, 50.564, 65.003

The generated product was determined as 1,9-bis[dimethyloctadecylammonium-hexafluorocyclopropane-1,3-bis(sulfonyl)imide]nonane from the spectra above.

Note that, in 1,9-bis[dimethyloctadecylammonium-hexafluorocyclopropane-1,3 bis(sulfonyl)imide]nonane, pKa of an acid that is a base of a conjugate base [hexafluorocyclopropane-1,3-bis(sulfonyl)imide] in acetonitrile is −0.8.

### Comparative Example 1

### Synthesis of 1-butyl-3-n-octadecylimiclazoliumhexafluorocyclopropane-1,3-bis(sulfonyl)imide

For comparison, synthesis of 1-butyl-3-n-octadecylimidazoliumhexafluorocyclopropane-1,3-bis(sulfonyl)imide that was a monocationic ionic liquid was performed according to the following scheme.

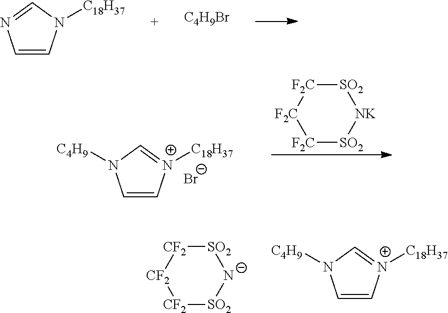

In acetonitrile, 10.7 g of 1-octadecylimidazole synthesized in Example 1 and 6.03 g of bromobuthane were dissolved, followed by heating the resulting solution to reflux for 5 hours. After removing the solvent, recrystallization was performed using a mixed solvent of n-hexane and ethanol to thereby obtain 1-butyl-3-octadecylimidazoliumbromide. The obtained bromide in an amount of 4.57 g was dissolved in ethanol. To the solution, an ethanol solution including 3.31 g of potassium hexafluorocyclopropane-1,3-bis(sulfonyl)imide was added, and the resultant was stirred. As a result, colorless precipitates were generated. The solution was heated to reflux for 1 hour. After cooling the solution, the solvent was removed. To the resultant, dichloromethane was added, and the dissolved part was filtered. The filtrate was dried, followed by performing recrystallization using a mixed solvent of n-hexane and ethanol to thereby obtain 6.00 g of colorless crystals of 1-butyl-3-n-octadecylimidazoliumhexafluorocyclopropane-1,3-bis(sulfonyl)imide. The yield was 90%.

The FTIR absorption of the generated product and the assignment are presented below.

Symmetric stretching vibrations of SO₂ were observed at 1,091 cm⁻¹, symmetric stretching vibrations of CF₂ were observed at 1,161 cm⁻¹, asymmetric stretching vibrations of a SO₂ bond were observed at 1,356 cm⁻¹, bending vibrations of CH₂ were observed at 1,470 cm⁻¹, stretching vibrations unique to imidazole were observed at 1,560 cm⁻¹, symmetric stretching vibrations of CH₂ were observed at 2,850 cm⁻¹, and asymmetric stretching vibrations of CH₂ were observed at 2,919 cm⁻¹.

Moreover, peaks of a proton (¹H)NMR and a carbon (¹³C)NMR in deuterated chloroform are presented below.

¹H-NMR (CDCl₃, δ ppm); 0.850 (t, 3H, J=7.2 Hz), 0.941 (t, 3H, J=7.2 Hz), 1.170-1.410 (m, 32H), 1.835 (quintet, 4H), 4.160 (m, 4H), 7.267 (d, 1H, J=2.1 Hz), 7.294 (d, 11-1, J=2.1 Hz), 8.749 (s, 1H)

¹³C-NMR (CDCl₃, δ ppm); 13.254, 14.085, 19.351, 22.663, 26.113, 28.853, 29.303, 29.333, 29.448, 29.570, 29.631, 29.677, 30.127, 31.898, 32.004, 49.977, 50.244, 122.179, 122.263, 135.473

The generated product was determined as 1-butyl-3-n-octadecylimidazolium hexafluorocyclopropane-1,3-bis(sulfonyl)imide from the spectra above.

Note that, in 1-butyl-3-n-octadecylimidazolium hexafluorocyclopropane-1,3-bis(sulfonyl)imide, pKa of an acid that is a base of a conjugate base [hexafluorocyclopropane-1,3-bis(sulfonyl)imide] in acetonitrile is −0.8.

### Comparative Example 2

### Synthesis of 1,9-bis[1-methylimidazolium bis(trifluoromethanesulfonyl)imide]nonane

Synthesis of 1,9-bis[1-methylimidazolium bis(trifluoromethanesulfonyl)imide]nonane that was an ionic liquid having a dication structure but not having a monovalent long-chain hydrocarbon group was performed according to the following scheme.

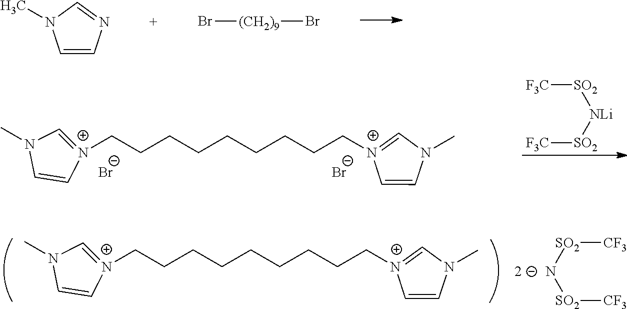

A dibromine salt was synthesized with reference to NPL [J. Am. Chem. Soc. Vol. 127 (2005) p. 593]. Specifically, 13.3 g of dibromononane was added little by little to 7.62 g of 1-methylimidazole at room temperature, followed by the resultant was allowed to react for 10 hours at room temperature. The resultant was dissolved in 100 mL of water, followed by washing with about 20 mL of ethyl acetate 3 times to separate an aqueous phase. After removing water from the aqueous phase by an evaporator, the resultant was further dried in a vacuum drier of 80° C. for 20 hours, to thereby obtain 19.9 g of a viscous liquid of a dibromine salt. The yield was 95%.

In 30 mL of water, 6.14 g of the bromide and a lithium sulfoimide were dissolved. To the solution, a solution obtained by dissolving 7.65 g of sulfoimide in 10 mL of water was added. After stirring the resultant for 10 hours at room temperature, an aqueous phase was removed, followed by washing with about 15 mL of water 3 times.

Moreover, peaks of a proton (¹H)NMR and a carbon (¹³C)NMR in CDCl₃ are presented below.

¹H-NMR (CDCl₃, δ ppm); 1.140-1.310 (m, 10H), 1.690-1.810 (m, 4H), 3.832 (s, 6H), 4.126 (t, 4H, J=7.2 Hz), 7.686 (d, 2H, J=1.5 Hz), 7.734 (d, 2H, J=1.5 Hz), 9.072 (s, 2H)

¹³C-NMR (CDCl₃, δ ppm); 25.673, 28.512, 28.817, 29.550, 35.900, 48.949, 122.410, 123.784, 136.635

The generated product was determined as 1,9-bis[1-methylimidazoliumbis(trifluoromethanesulfonyl)imide]nonane from the spectra above.

Note that, in 1,9-bis[1-methylimidazolium bis(trifluoromethanesulfonyl)imide]nonane, pKa of an acid that is a base of a conjugate base [bis(trifluoromethanesulfonyl)imide] in acetonitrile is 0.3.

### Comparative Example 3

### Synthesis of 1,9-bis[1-methylimidazolium bis(nonafluorobutanesulfonyl)imide]nonane

Synthesis of 1,9-bis[1-methylimidazolium bis(nonafluorobutanesulfonyl)imide]nonane that was an ionic liquid having a dication structure but not having a monovalent long-chain hydrocarbon group was performed according to the following scheme.

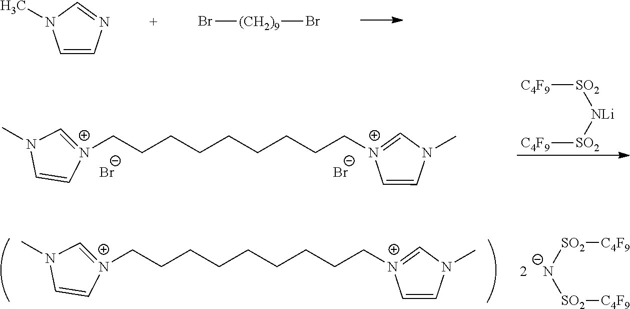

In 60 mL of Water, 3.63 g of bromide synthesized in Comparative Example 2 was dissolved. To the solution, a solution obtained by dissolving sulfoimide in a mixed solution of water and ethanol was added. After reacting the resultant for 10 hours at room temperature, the solvent was removed, followed by washing with 15 mL of water 3 times. After confirming that the silver nitrate test indicated negative, the resultant was dried for 24 hours at 80° C.

The FTIR absorption of the generated product and the assignment are presented below.

Symmetric stretching vibrations of SO₂ were observed at 1,074 cm⁻¹, symmetric stretching vibrations of CF₂ were observed at 1,169 cm⁻¹ asymmetric stretching vibrations of a SO₂ bond were observed at 1,352 cm⁻¹, bending vibrations of CH₂ were observed at 1,471 cm⁻¹, stretching vibrations unique to imidazole were observed at 1,574 cm⁻¹, symmetric stretching vibrations of CH₂ were observed at 2,864 cm⁻¹, and asymmetric stretching vibrations of CH₂ were observed at 2,937 cm⁻¹.

Moreover, peaks of a proton (¹H)NMR and a carbon (¹³C)NMR in CDCl₃ are presented below.

¹H-NMR (CDCl₃, δ ppm); 1.140-1.280 (m, 10H), 1.700-1.800 (m, 4H), 3.830 (s, 6H), 4.124 (t, 4H, J=7.2 Hz), 7.681 (d, 2H, J=1.5 Hz), 7.733 (d, 2H, J=1.5 Hz), 9.071 (s, 2H)

¹³C-NMR (CDCl₃, δ ppm); 25.673, 28.512, 28.817, 29.550, 35.884, 48.934, 122.410, 123.784, 136.635

The generated product was determined as 1,9-bis[1-methylimidazolium bis(nonafluorobutanesulfonyl)imide]nonane from the spectra above.

Note that, in 1,9-bis[1-methylimidazolium bis(nonafluorobutanesulfonyl)imide]nonane, pKa of an acid that is a base of a conjugate base [bis(nonafluorobutanesulfonyl)imide] in acetonitrile is 0.0.

### Comparative Example 4

### Synthesis of 1,9-bis[1,8-diazabicyclo[5.4.0]-7-undeceniumhexafluorocyclopropane-1,3-bis(sulfonyl)imide]nonane

Synthesis of 1,9-bis[1,8-diazabicyclo[5.4.0]-7-undeceniumhexafluorocyclopropane-1,3-bis(sulfonyl)imide]nonane was performed according to the following scheme.

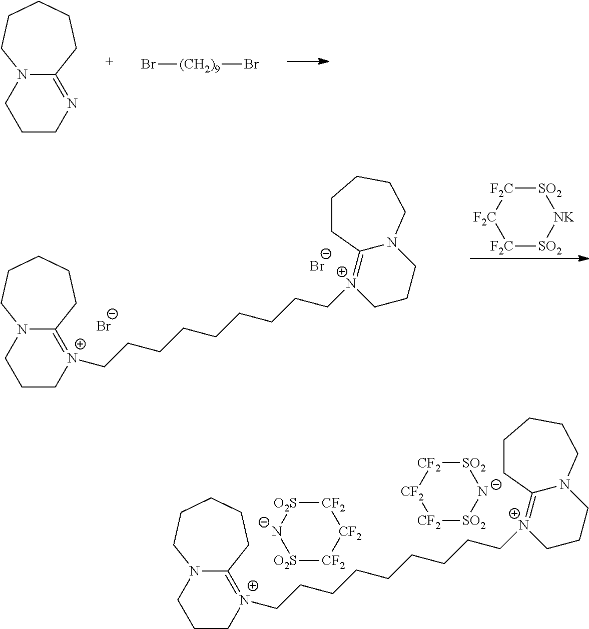

1,8-Diazabicyclo[5.4.0]-7-undecene and 1,9-dibromononane that was 0.5 times a mole of the 1,8-diazabicyclo[5.4.0]-7-undecene were mixed, and the resultant was stirred. When the heat generation was subsided, heating was performed to make the internal temperature of the flask 110° C., and the mixture was stirred for 3 hours. After completing the reaction, impurities and unreacted products were extracted with ethyl acetate. Thereafter, acetone was added to precipitate crystals, followed by collecting the crystals through filtration, and drying the crystals. The yield was 80%.

The obtained dibromo compound was dissolved in water. To the solution, a solution obtained by dissolving potassium hexafluorocyclopropane-1,3-bis(sulfonyl)imide that was 2 times a mole of the dibromine compound in water and a small amount of ethanol was added, followed by stirring for 1 hour. Thereafter, the resultant was heated to reflux for 1 hour. After cooling, the solvent was removed, extraction was performed with dichloromethane, and the resultant was dried with anhydrous magnesium sulfate, followed by removing the solvent. Then, vacuum drying was performed for 12 hours at 100° C. The yield was 96%.

The FTIR absorption of the generated product and the assignment are presented below.

Asymmetric stretching vibrations of SNS were observed at 1,036 cm⁻¹, symmetric stretching vibrations of SO₂ were observed at 1,088 cm⁻¹, symmetric stretching vibrations of CF₂ were observed at 1,153 cm⁻¹, asymmetric stretching vibrations of SO₂ were observed at 1,352 cm⁻¹, stretching vibrations of C═N were observed at 1,618 cm⁻¹, symmetric stretching vibrations of CH₂ were observed at 2,860 cm⁻¹, and asymmetric stretching vibrations of CH₂ were observed at 2,933 cm⁻¹.

Moreover, peaks of a proton (¹H)NMR and a carbon (¹³C)NMR in CD₃OD are presented below.

¹H-NMR (CD₃OD, δ ppm); 1.300-1.400 (m, 10H), 1.630-1.670 (m, 4H), 1.700-1.810 (m, 12H), 2.030-2.100 (m, 4H), 2.850-2.880 (m, 4H), 3.490-3.540 (m, 12H), 3.630-3.660 (m, 4H)

¹³C-NMR (CD₃OD, δ ppm); 21.038, 24.182, 27.021, 27.387, 29.005, 29.601, 29.646, 30.210, 30.271, 50.111, 55.014, 55.915, 167.910

The generated product was determined as 1,9-bis[1,8-diazabicyclo[5.4.0]-7-undeceniumhexafluorocyclopropane-1,3-bis(sulfonyl)imide]nonane from the spectra above.

Note that, in 1,9-bis[1,8-diazabicyclo[5.4.0]-7-undeceniumhexafluorocyclopropane-1,3-bis(sulfonyl)imide]nonane, pKa of an acid that is a base of a conjugate base [hexafluorocyclopropane-1,3-bis(sulfonyl)imide] in acetonitrile is −0.8.

### Comparative Example 5

### Synthesis of 1-butyl-3-n-octadecylimidazoliumbis(nonafluorobutanesulfonyl)imide

For comparison, synthesis of 1-butyl-3-n-octadecylimidazoliumbis(nonafluorobutanesulfonyl)imide that was a monocation ionic liquid was performed according to the following scheme.

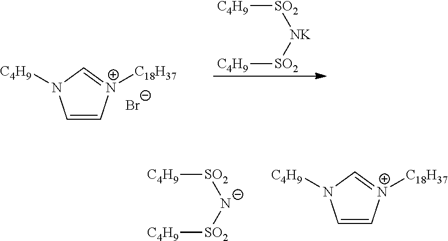

In ethanol, 1.27 g of 1-butyl-3-octadecylimidazoliumbromide synthesized in Comparative Example 1 was dissolved. To the solution, an ethanol solution including 1.81 g of potassium bis(nonafluorobutanesulfonyl)imide was added, followed by stirring. As a result, colorless precipitates were generated. The solution was heated to reflux for 1 hour. After cooling the solution, the solvent was removed. To the resultant, dichloromethane was added, and the soluble part was subjected to filtration. The filtrate was dried, and the resultant was subjected to recrystallization using a mixed solvent of n-hexane and ethanol to thereby obtain 2.06 g of colorless crystals of 1-butyl-3-n-octadecylimidazoliumbis(nonafluorobutanesulfonyl)imide. The yield was 74.9%.

The FTIR absorption of the generated product and the assignment are presented below.

Symmetric stretching vibrations of SO₂ were observed at 1,072 cm⁻¹, symmetric stretching vibrations of CF were observed at 1,137 cm⁻¹ and 1,168 cm⁻¹, asymmetric stretching vibrations of a SO₂ bond were observed at 1,352 cm⁻¹, bending vibrations of CH₂ were observed at 1,469 cm⁻¹, CN stretching vibrations of imidazole were observed at 1,564 cm⁻¹, symmetric stretching vibrations of CH₂ were observed at 2,850 cm⁻¹, and asymmetric stretching vibrations of CH₂ were observed at 2,920 cm⁻¹.

Moreover, peaks of a proton (¹H)NMR and a carbon (¹³C)NMR in deuterated DMSO are presented below.

¹H-NMR (deuterated DMSO, δ ppm); 0.837 (t, 3H, J=7.2 Hz), 0.885 (t, 3H, J=6.6 Hz), 1.140-1.300 (m, 32H), 1.760 (quintet, 4H), 4.112-4.173 (m, 4H), 7.776 (s, 1H), 7.781 (s, 1H), 9.175 (s, 1H)

¹³C-NMR (deuterated DMSO, δ ppm); 13.341, 14.043, 18.927, 22.239, 25.627, 28.466, 28.863, 28.970, 29.062, 29.184, 29.413, 31.443, 48.751, 49.026, 122.624, 136.101

The generated product was determined as 1-butyl-3-n-octadecylimidazoliumbis(nonafluorobutanesulfonyl)imide from the spectra above.

Note that, in 1-butyl-3-n-octadecylimidazoliumbis(nonafluorobutanesulfonyl)imide, pKa of an acid that is a base of a conjugate base [bis(nonafluorobutanesulfonyl)imide] in acetonitrile is 0.0.

### Example 16

### <Thermal Stability Measurement Results>

The 5% weight reduction temperature, 10% weight reduction temperature, and 20% weight reduction temperature of 1,9-bis[1-octadecylimidazolium bis(trifluoromethanesulfonyl)imide]nonane synthesized in Example 1 were 300.2° C., 319.3° C., and 373.0° C., respectively. Compared to the commercial products presented as Comparative Examples which were known as common lubricants used for magnetic recording media, it was found that the 5% weight reduction temperature, 10% weight reduction temperature, and 20% weight reduction temperature were higher than perfluoropolyether Z-DOL (Comparative Example 11) by 100° C. or greater, and higher than Z-TETRAOL (Comparative Example 12) by 50° C. or higher.

### Example 17

### <Thermal Stability Measurement Results>

The 5% weight reduction temperature, 10% weight reduction temperature, and 20% weight reduction temperature of 1,9-bis[1-octadecylimidazolium hexafluorocyclopropane-1,3-bis(sulfonyl)imide]nonane synthesized in Example 2 were 367.7° C., 392.7° C., and 416.2° C., respectively. Compared to 1-butyl-3-n-octadecylimidazoliumhexafluorocyclopropane-1,3-bis(sulfonyl)imide of Comparative Example 1 that was a monocation, the 5% weight reduction temperature, 10% weight reduction temperature, and 20% weight reduction temperature were higher. It is assumed that it is because of an effect of the dication.

Compared to commercial products of perfluoropolyether Z-DOL (Comparative Example 11) and Z-TETRAOL (Comparative Example 12), moreover, it was found that thermal stability was significantly improved.

### Example 18

### <Thermal Stability Measurement Results>

The 5% weight reduction temperature, 10% weight reduction temperature, and 20% weight reduction temperature of 1,9-bis(1-octadecylimidazolium tricyanomethanide)nonane synthesized in Example 3 were 313.6° C., 326.8° C., and 344.0° C., respectively. Compared to commercial products of perfluoropolyether Z-DOL (Comparative Example 11) and Z-TETRAOL (Comparative Example 12), it was found that thermal stability was improved.

### Example 19

### <Thermal Stability Measurement Results>

The 5%© weight reduction temperature, 10% weight reduction temperature, and 20% weight reduction temperature of 1,5-bis[1-octadecylimidazolium bis(trifluoromethanesulfonyl)imide]pentane synthesized in Example 4 were 344.2° C., 383.1° C., and 411.6° C., respectively. Compared to the commercial products presented as Comparative Examples which were known as common lubricants used for magnetic recording media, if was found that the 5% weight reduction temperature, 10% weight reduction temperature, and 20% weight reduction temperature were higher than perfluoropolyether Z-DOL (Comparative Example 11) by 150° C. or greater, and higher than Z-TETRAOL (Comparative Example 12) by 100° C. or higher.

### Example 20

### <Thermal Stability Measurement Results>

The 5% weight reduction temperature, 10% weight reduction temperature, and 20% weight reduction temperature of 1,5-bis[1-octadecylimidazolium hexafluorocyclopropane-1,3-bis(sulfonyl)imide]pentane synthesized in Example 5 were 362.3° C., 397.8° C., and 423.5° C., respectively. Compared to 1-butyl-3-n-octadecylimidazoliumhexafluorocyclopropane-1,3-bis(sulfonyl)imide of Comparative Example 1 that was a monocation, the 5% weight reduction temperature, 10% weight reduction temperature, and 20% weight reduction temperature were higher. It is assumed that it is because of an effect of the dication.

Compared to commercial products of perfluoropolyether Z-DOL (Comparative Example 11) and Z-TETRAOL (Comparative Example 12), moreover, it was found that thermal stability was significantly improved.

### Example 21

### <Thermal Stability Measurement Results>

The 5% weight reduction temperature, 10% weight reduction temperature, and 20% weight reduction temperature of 1,5-bis[1-octadecylimidazolium-bis(nonafluorobutanesulfonyl)imide)pentane synthesized in Example 6 were 359.9° C., 383.4° C., and 411.5° C., respectively. Compared to commercial products of perfluoropolyether Z-DOL (Comparative Example 11) and Z-TETRAOL (Comparative Example 12), it was found that thermal stability was improved.

### Example 22

### <Thermal Stability Measurement Results>

The 5% weight reduction temperature, 10% weight reduction temperature, and 20% weight reduction temperature of 1,9-bis[1-octadecylpyrrolidinium bis(trifluoromethanesulfonyl)imide]nonane synthesized in Example 7 were 303.6° C., 356.0° C., and 377.8° C., respectively. Compared to commercial products of perfluoropolyether Z-DOL (Comparative Example 1.1) and Z-TETRAOL (Comparative Example 12), it was found that thermal stability was improved.

### Example 23

### <Thermal Stability Measurement Results>

The 5% weight reduction temperature, 10% weight reduction temperature, and 20% weight reduction temperature of 1,9-bis[1-octadecylpyrrolidinium hexafluorocyclopropane-1,3-bis(sulfonyl)imide]nonane synthesized in Example 8 were 333.9° C., 373.4° C., and 395.7° C., respectively. Compared to commercial products of perfluoropolyether Z-DOL (Comparative Example 11) and Z-TETRAOL (Comparative Example 12), it was found that thermal stability was improved.

### Example 24

### <Thermal Stability Measurement Results>

The 5% weight reduction temperature, 10% weight reduction temperature, and 20% weight reduction temperature of 1,9-bis[6-octadecyl-1,8-diazabicyclo[5.4.0]-7-undeceniumhexafluorocyclopropane-1,3-bis (sulfonyl)imide]nonane synthesized in Example 9 were 410.2° C., 426.2° C., and 445.8° C., respectively. Compared to commercial products of perfluoropolyether Z-DOL (Comparative Example 11) and Z-TETRAOL (Comparative Example 12), it was found that thermal stability was improved.

### Example 25

### <Thermal Stability Measurement Results>

The 5% weight reduction temperature, 10% weight reduction temperature, and 20% weight reduction temperature of 1,3-bis[1-octadecylimidazolium bis(nonafluorobutanesulfonyl)imide]propane synthesized in Example 10 were 352.3° C., 381.9° C., and 401.4° C., respectively. Compared to the commercial products presented as Comparative Examples which were known as common lubricants used for magnetic recording media, it was found that the 5% weight reduction temperature, 10% weight reduction temperature, and 20% weight reduction temperature were higher than perfluoropolyether Z-DOL (Comparative Example 11) by 100° C. or greater, and higher than Z-TETRAOL (Comparative Example 12) by 50° C. or higher.

### Example 26

### <Thermal Stability Measurement Results>

The 5% weight reduction temperature, 10% weight reduction temperature, and 20% weight reduction temperature of 1,3-bis[1-octadecylimidazolium hexafluorocyclopropane-1,3-bis(sulfonyl)imide]propane synthesized in Example 11 were 342.9° C., 382.4° C., and 412.7° C., respectively. Compared to 1-butyl-3-n-octadecylimidazoliumhexafluorocyclopropane-1,3-bis(sulfonyl)imide of Comparative Example 1 that was a monocation, the 5% weight reduction temperature, 10% weight reduction temperature, and 20% weight reduction temperature were higher. It is assumed that it is because of an effect of the dication.

Compared to commercial products of perfluoropolyether Z-DOL (Comparative Example 11) and Z-TETRAOL (Comparative Example 12), moreover, it was found that thermal stability was significantly improved.

### Example 27

### <Thermal Stability Measurement Results>

The 5% weight reduction temperature, 10% weight reduction temperature, and 20%© weight reduction temperature of 1,9-bis[1-octadecylimidazolium bis(nonafluorobutanesulfonyl)imide]nonane synthesized in Example 12 were 355.1° C., 381.0° C., and 400.9° C., respectively. Compared to Comparative Example 1, the 5% weight reduction temperature, 10% weight reduction temperature, and 20% weight reduction temperature were high. It is assumed that it is because of an effect of the dication. Compared to commercial products of perfluoropolyether Z-DOL (Comparative Example 11) and Z-TETRAOL (Comparative Example 12), it was found that thermal stability was improved.

### Example 28

### <Thermal Stability Measurement Results>

The 5% weight reduction temperature, 10% weight reduction temperature, and 20% weight reduction temperature of 1,9-bis[1-octadecylimidazoliumnonafluorobutanesulfonium]nonane synthesized in Example 13 were 348.7° C., 372.0° C., and 392.1° C., respectively. Compared to the commercial products presented as Comparative Examples which were known as common lubricants used for magnetic recording media, it was found that the 5% weight reduction temperature, 10% weight reduction temperature, and 20% weight reduction temperature were higher than perfluoropolyether Z-DOL (Comparative Example 11) by 150° C. or greater, and higher than Z-TETRAOL (Comparative Example 12) by 100° C. or higher. Compared to Comparative Example 1, the 5% weight reduction temperature was high, but the 10% weight reduction temperature and 20% weight reduction temperature were low. This is because a perfluorosulfonic acid salt is typically has a low decomposition temperature compared to sulfonylimide.

### Example 29

### <Thermal Stability Measurement Results>

The 5% weight reduction temperature, 10% weight reduction temperature, and 20% weight reduction temperature of 1,9-bis[dimethyloctadecylammonium-bis(nonafluorobutanesulfonyl)imide]nonane synthesized in Example 14 were 334.1° C., 365.1° C., and 386.6° C., respectively. Compared to commercial products of perfluoropolyether Z-DOL (Comparative Example 11) and Z-TETRAOL (Comparative Example 12), it was found that thermal stability was significantly improved.

### Example 30

### <Thermal Stability Measurement Results>

The 5% weight reduction temperature, 10% weight reduction temperature, and 20% weight reduction temperature of 1,9-bis[dimethyloctadecylammonium-hexafluorocyclopropane-1,3-bis(sulfonyl)imide]nonane synthesized in Example 15 were 330.6° C., 369.6° C., and 392.5° C., respectively. Compared to commercial products of perfluoropolyether Z-DOL (Comparative Example 11) and Z-TETRAOL (Comparative Example 12), it was found that thermal stability was improved.

### Comparative Example 6

### <Thermal Stability Measurement Results>

The 5% weight reduction temperature, 10% weight reduction temperature, and 20% weight reduction temperature of 1-butyl-3-n-octadecylimidazoliumhexafluorocyclopropane-1,3-bis(sulfonyl)imide, which was synthesized in Comparative Example 1 and was a monocation ionic liquid, were 342.8° C., 372.6° C., and 400.8° C., respectively. The thermal stability was high because it was the ionic liquid.

### Comparative Example 7

### <Thermal Stability Measurement Results>

The 5% weight reduction temperature, 10%© weight reduction temperature, and 20% weight reduction temperature of 1,9-bis[1-methylimidazolium bis(trifluoromethanesulfonyl)imide]nonane, which was synthesized in Comparative Example 2 and was an ionic liquid that was dication and having no long-chain hydrocarbon, were 392.3° C., 410.6° C., and 472.3° C., respectively. The thermal stability was significantly high because it was the dication-based ionic liquid.

### Comparative Example 8

### <Thermal Stability Measurement Results>

The 5% weight reduction temperature, 10% weight reduction temperature, and 20% weight reduction temperature of 1,9-bis[1-methylimidazolium bis(nonafluorobutanesulfonyl)imide]nonane, which was synthesized in Comparative Example 3 and was an ionic liquid that was dication and had no long-chain hydrocarbon, were 380.4° C., 394.5° C., and 406.4° C., respectively. The thermal stability was significantly high because it was the dication-based ionic liquid.

### Comparative Example 9

### <Thermal Stability Measurement Results>

The 5% weight reduction temperature, 10% weight reduction temperature, and 20% weight reduction temperature of 1,9-bis[1,8-diazabicyclo[5.4.0]-7-undeceniumhexafluorocyclopropane-1,3-bis(sulfonyl)imide]nonane, which was synthesized in Comparative Example 4 and was an ionic liquid that was dication and had no long-chain hydrocarbon, were 415.6° C., 432.6° C., and 451.3° C., respectively. The thermal stability was improved because it was the dication-based ionic liquid.

### Comparative Example 10

### <Thermal Stability Measurement Results>

The 5% weight reduction temperature, 10% weight reduction temperature, and 20% weight reduction temperature of 1-butyl-3-n-octadecylimidazoliumbisnonafluorobutane (sulfonyl)imide, which was synthesized in Comparative Example 5 and was a monocation ionic liquid, were 333.1° C., 367.6° C., 393.3° C., respectively. The thermal stability was improved because it was the dication-based ionic liquid.

### Comparative Example 11

### <Thermal Stability Measurement Results>

Perfluoropolyether (Z-DOL) having a hydroxyl group at a terminal and having a molecular weight of about 2,000, which was a commercial product and was typically used as a lubricant for magnetic recording media, was used as a lubricant of Comparative Example 11. The 5% weight reduction temperature, 10% weight reduction temperature, and 20% weight reduction temperature of Z-DOL were 165.0° C., 197.0° C., and 226.0° C., respectively. The weight reduction was caused due to evaporation.

### Comparative Example 12

### <Thermal Stability Measurement Results>

Perfluoropolyether (Z-TETRAOL) having a plurality of hydroxyl groups at a terminal and having a molecular weight of about 2,000, which was a commercial product and was typically used as a lubricant for magnetic recording media, was used as a lubricant of Comparative Example 12. The 5% weight reduction temperature, 10% weight reduction temperature, and 20% weight reduction temperature of Z-TETRAOL were 240.0° C., 261.0° C., and 282.0° C., respectively, and the main endothermic temperature at the time of decomposition was 386.3° C. Similarly to Z-DOL, the weight seduction was caused due to evaporation.

The results of Examples 16 to 30 and Comparative Examples 6 to 12 are summarized in Tables 2-1 and 2-2.

TABLE 2-1

| | | | Weight reduction | | |
|---|---|---|---|---|---|
| | Synthesis | | temperature (° C.) | | |
| Example | Example | Compound name | 5% | 10% | 20% |
| Ex. 16 | Ex. 1 | 1,9-bis[1-octadecylimidazolium | 300.2 | 319.3 | 373.0 |
| | | bis(trifluoromethanesulfonyl)imide]nonane | | | |
| Ex. 17 | Ex. 2 | 1,9-bis[1-octadecylimidazolium | 367.7 | 392.7 | 416.2 |
| | | hexafluorocyclopropane-1,3-bis | | | |
| | | (sulfonyl)imide]nonane | | | |
| Ex. 18 | Ex. 3 | 1,9-bis(1-octadecylimidazolium | 313.6 | 326.8 | 344.0 |
| | | tricyanomethide)nonane | | | |
| Ex. 19 | Ex. 4 | 1,5-bis[1-octadecylimidazolium | 344.2 | 383.1 | 411.6 |
| | | bis(trifluoromethanesulfonyl)imide] | | | |
| | | pentane | | | |
| Ex. 20 | Ex. 5 | 1,5-bis[1-octadecylimidazolium | 362.3 | 397.8 | 423.5 |
| | | hexafluorocyclopropane- | | | |
| | | 1,3-bis(sulfonyl)imide]pentane | | | |
| Ex. 21 | Ex. 6 | 1,5-bis[1-octadecylimidazolium- | 359.9 | 383.4 | 411.5 |
| | | bis(nonafluorobutanesulfonyl)imide] | | | |
| | | pentane | | | |
| Ex. 22 | Ex. 7 | 1,9-bis[1-octadecylpyrrolidinium | 303.6 | 356.0 | 377.8 |
| | | bis(trifluoromethanesulfonyl)imide] | | | |
| | | nonane | | | |
| Ex. 23 | Ex. 8 | 1,9-bis[1-octadecylpyrrolidinium | 333.9 | 373.4 | 395.7 |
| | | hexafluorocyclopropane- | | | |
| | | 1,3-bis(sulfonyl)imide]nonane | | | |
| Ex. 24 | Ex. 9 | 1,9-bis[6-octadecyl-1,8- | 410.2 | 426.2 | 445.8 |
| | | diazabicyclo[5.4.0]-7-undecenium | | | |
| | | hexafluorocyclopropane- | | | |
| | | 1,3-bis(sulfonyl)imide]nonane | | | |
| Ex. 25 | Ex. 10 | 1,3-bis[1-octadecylimidazolium | 352.3 | 381.9 | 401.4 |
| | | bis(nonafluorobutanesulfonyl)imide] | | | |
| | | propane | | | |
| Ex. 26 | Ex. 11 | 1,3-bis[1-octadecylimidazolium | 342.9 | 382.4 | 412.7 |
| | | hexafluorocyclopropane- | | | |
| | | 1,3-bis(sulfonyl)imide]propane | | | |
| Ex. 27 | Ex. 12 | 1,9-bis[1-octadecylimidazolium | 355.1 | 381.0 | 400.9 |
| | | bis(nonafluorobutanesulfonyl)imide] | | | |
| | | nonane | | | |
| Ex. 28 | Ex. 13 | 1,9-bis[1-octadecylimidazolium | 348.7 | 372.0 | 392.1 |
| | | nonafluorobutanesulfonium]nonane | | | |
| Ex. 29 | Ex. 14 | 1,9-bis[dimethyloctadecylammonium- | 334.1 | 365.1 | 386.6 |
| | | bis(nonafluorobutanesulfonyl)imide] | | | |
| | | nonane | | | |
| Ex. 30 | Ex. 15 | 1,9-bis[dimethyloctadecylammonium- | 330.6 | 369.6 | 392.5 |
| | | hexafluorocyclopropane- | | | |
| | | 1,3-bis(sulfonyl)imide]nonane | | | |

TABLE 2-2

| | | | Weight reduction | | |
|---|---|---|---|---|---|
| | Synthesis | | temperature (° C.) | | |
| Example | Example | Compound name | 5% | 10% | 20% |
| Comp. | Comp. | 1-butyl-3-n-octadecylimidazolium | 342.8 | 372.6 | 400.8 |
| Ex. 6 | Ex. 1 | hexafluorocyclopropane- | | | |
| | | 1,3-bis(sulfonyl)imide | | | |
| Comp. | Comp. | 1,9-bis[1-methylimidazolium | 392.3 | 410.6 | 472.3 |
| Ex. 7 | Ex. 2 | bis(trifluoromethanesulfonyl)imide] | | | |
| | | nonane | | | |
| Comp. | Comp. | 1,9-bis[1-methylimidazolium | 380.4 | 394.5 | 406.4 |
| Ex. 8 | Ex. 3 | bis(nonafluorobutanesulfonyl)imide] | | | |
| | | nonane | | | |
| Comp. | Comp. | 1,9-bis[1,8-diazabicyclo[5.4.0]- | 415.6 | 432.6 | 451.3 |
| Ex. 9 | Ex. 4 | 7-undecenium hexafluorocyclopropane- | | | |
| | | 1,3-bis(sulfonyl)imide]nonane | | | |
| Comp. | Comp. | 1-butyl-3-n-octadecylimidazolium | 333.1 | 367.6 | 393.3 |
| Ex. 10 | Ex. 5 | bis(nonafluorobutanesulfonyl)imide | | | |

| | | | | |
|---|---|---|---|---|
| Comp. Ex. 11 | Z-DOL | 165.0 | 197.0 | 226.0 |
| Comp. Ex. 12 | Z-Tetraol | 240.0 | 261.0 | 282.0 |

As described above, the ionic liquid-based lubricants had excellent thermal stability compared to the perfluoropolyether of the commercial products of Comparative Examples 11 and 12.

Moreover, the ionic liquid of Comparative Example 1 had a structure where the bond chain of the ionic liquid of Examples 2 and 5 was cut at the middle. It was found by comparing the thermal stability of Examples 16 and 19 to Comparative Example 6, use of the dication improved the 5%, 10%, and 20% weight reduction temperatures by nearly 20° C. The bis(trifluoromethanesulfonyl)imide or bis(nonafluorobutanesulfonyl)imide salt, which generally has the poorer thermal stability than hexafluorocyclopropane-1,3-bis(sulfonyl)imide, improved thermal stability compared to Comparative Example, hence an effect obtained by using the dication was exhibited.

Moreover, it was found by comparing the weight reduction temperatures of Examples 16, 19, and 21 to the weight reduction temperatures of Comparative Examples 7 and 8 that, among dications, Examples 16, 19, and 21 each having a long-chain alkyl chain had low thermal stability compared to Comparative Examples 7 and 8 each having a short alkyl chain.

Moreover, the cation sites of the ionic liquids of Comparative Examples 6 and 10 each had a structure similar to a compound where the bonding chain of the ionic liquid of each of Examples 25 to 28 was cut at the middle. In the case where the anion site was hexafluorocyclopropane-1,3-bis(sulfonyl)imide, Example 26 and Comparative Example 6 could be compared. It was found that the 5% and 10% weight reduction temperatures were improved by 10° C. or greater.

In the case where the anion site was bis(nonafluorobutanesulfonyl)imide, moreover, Examples 25 and 27 and Comparative Example 10 could be compared. It was found that in the case of Example 25 and in the case of Example 27, the 5% and 10% weight reduction temperatures were improved by 15° C. or greater. It was found from the above that, in terms of the thermal stability, the weight reduction temperatures were improved by using the dication, compared to the monocation.

Moreover, it was found by comparing the weight reduction temperatures of Examples 25, 26, 27, and 28 to the weight reduction temperatures of Comparative Examples 7 and 8 that, among dications, Examples 25 to 28 each having a long-chain alkyl chain had low thermal stability compared to Comparative Examples 7 and 8 each having a short alkyl chain.

It is suggested in NPL (Thermochim. Acta, 1991, Vol. 185, pp. 1-11) that the thermal stability reduces as an alkyl chain becomes longer. Specifically, it is assumed that the thermal stability was reduced because the reverse Menshutkin reaction of the alkyl chain was caused. However, the antifriction properties were significantly improved, when the long-chain alkyl chain was included, as described later.

### Example 31

### <Disk Durability Test 1>

A friction test was performed with the lubricant of Example 1 by means of the pin-on-disk test machine illustrated in FIG. 4. The result is depicted in FIG. 5.

### Comparative Example 13

### <Disk Durability Test 1>

A friction test was performed with the lubricant of Comparative Example 1 in the same manner as in Example 31. The result is depicted in FIG. 5.

### Comparative Example 14

### <Disk Durability Test 1>

A friction test was performed with the lubricant of Comparative Example 2 in the same manner as in Example 31. The result is depicted in FIG. 5.

### Comparative Example 15

### <Disk Durability Test 1>

A friction test was performed with Z-DOL that was a commercially available lubricant in the same manner as in Example 31. The result is depicted in FIG. 5.

As presented with Comparative Example 15, in the pin-on-disk test, the friction coefficient of Z-DOL that was the commercially available lubricant was stably low.

As presented with Example 31, the friction coefficient of the dication lubricant was stably low, and was low in forwards and backwards sliding motions of 100 times compared to Z-DOL of the commercial product.

The monocation of Comparative Example 1 had a long-chain alkyl chain, but the friction coefficient was high compared to that of dication or Z-DOL of the commercial product (Comparative Example 13). As presented with Comparative Example 14, the friction coefficient was extremely high with the ionic liquid that was the dication ionic liquid and did not include a long-chain alkyl chain.

It was found from the above that in the typical pin-on-disk test, the dication ionic liquid including a long-chain alkyl chain had a stably low friction coefficient compared to the ionic liquid that was monocation or did not include a long-chain alkyl chain, and moreover the friction coefficient of the dication ionic liquid including a long-chain alkyl chain was low compared to commercially available Z-DOL.

### Example 32

### <Disk Durability Test 1>

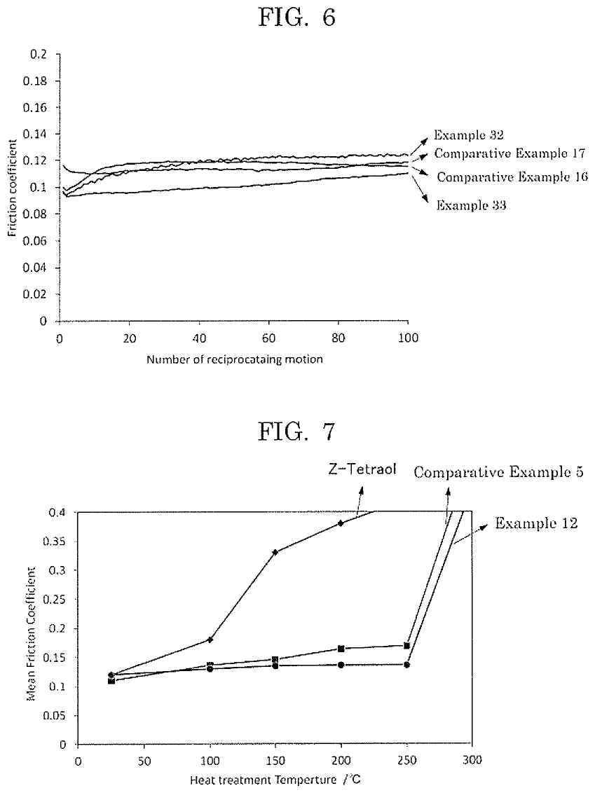

A friction test was performed with the lubricant of Example 12 in the same manner as in Example 31. The result is depicted in FIG. 6.

### Example 33

### <Disk Durability Test 1>

A friction test was performed with the lubricant of Example 13 in the same manner as in Example 31. The result is depicted in FIG. 6.

### Comparative Example 16

### <Disk Durability Test 1>

A friction test was performed with the lubricant of Comparative Example 2 in the same manner as in Example 31. The result is depicted in FIG. 6.

### Comparative Example 17

### <Disk Durability Test 1>

A friction test was performed with Z-Tetraol that was a commercially available lubricant in the same manner as in Example 31. The result is depicted in FIG. 6. As presented in Example 32, Example 33, Comparative Example 16, and

Comparative Example 17, in the pin-on-disk tests at 25° C., the friction coefficient of any of Z-Tetraol that was the commercially available lubricant, the dication-based ionic liquid, and the monocation-based ionic liquid was stably low. Among them, Example 33 had the lowest friction coefficient.

Next, a heat test was performed under the following temperature conditions, followed by performing a friction test using a pin-on-disk test as illustrated in FIG. 4 in the same manner as in Example 32. The results are summarized in Table 3 below and FIG. 7.

Specifically, media to each of which a lubricant including each of samples described in Table 3 was heated for 10 minutes to prepare samples. The samples are subjected to a friction test in the same manner as in Example 32. A friction coefficient at the time of 100th forwards and backwards motion was determined.

TABLE 3

| | Heating | |
|---|---|---|
| | temperature | Friction |
| Sample | (° C.) | coefficient |
| Ex. 12 | 100 | 0.130 |
| | 150 | 0.135 |
| | 200 | 0.136 |
| | 250 | 0.139 |
| Comp. | 100 | 0.136 |
| Ex. 5 | 150 | 0.146 |
| | 200 | 0.164 |
| | 250 | 0.169 |
| Z- | 100 | 0.180 |
| Tetraol | 150 | 0.330 |
| | 200 | 0.380 |
| | 250 | 0.430 |

As it was found from Table 3 and FIG. 7, the media to which Z-Tetraol was applied significantly increased the friction coefficients thereof when the temperature was greater than 100° C., and the lubrication effect was lost. On the other hand, the friction coefficient of the ionic liquid-based lubricant did not increase after heating up to 250° C. The friction coefficients of the monocation and dication were 0.136 (monocation) and 0.130 (dication) after heating at 100° C., 0.146 (monocation) and 0.135 (dication) after heating at 150° C., 0.164 (monocation) and 0.136 (dication) after heating at 200° C., and 0.169 (monocation) and 0.139 (dication) after heating at 250° C. The friction coefficient of the monocation started to increase at a temperature around or higher than 150° C., whereas the friction coefficient of the dication hardly increased until after heating at 250° C. Specifically, it was considered that the friction coefficient after the heat test was maintained low because the thermal stability was improved because of use of the dication.

It was found from the above that in the typical pin-on-disk test, the friction coefficient of the dication ionic liquid was significantly improved compared to commercially available Z-Tetraol. Moreover, the dication had a low friction coefficient after the heat test compared to the monocation ionic liquid, and the effect of the dication was exhibited.

### Examples 34 to 48

The above-described magnetic disk was produced using a lubricant including an ionic liquid of each of Examples 1 to 15. As presented in Table 4, the CSS measurement of the magnetic disk was greater than 50,000 times, and the CSS measurement after the heat test was also greater than 50,000 times, hence excellent durability was exhibited.

### Comparative Example 18

### <Disk Durability Test 2>

The above-described magnetic disk was produced using a lubricant including the ionic liquid of Comparative Example 1. As presented in Table 4, the CSS measurement of the magnetic disk was greater than 50,000 times, and the CSS measurement after the heat test was also greater than 50,000 times, hence excellent durability was exhibited.

### Comparative Example 19

### <Disk Durability Test 2>

The above-described magnetic disk was produced using a lubricant including the ionic liquid of Comparative Example 2. As presented in Table 4, the CSS measurement of the magnetic disk was greater than 13,200 times, and the CSS measurement after the heat test was 8,965 times, hence the result of durability was insufficient.

### Comparative Example 20

### <Disk Durability Test 2>

The above-described magnetic disk was produced using a lubricant including the ionic liquid of Comparative Example 3. As presented in Table 4, the CSS measurement of the magnetic disk was greater than 12,350 times, and the CSS measurement after the heat test was 8,700 times, hence the result of durability was insufficient.

### Comparative Example 21

### <Disk Durability Test 2>

The above-described magnetic disk was produced using a lubricant including the ionic liquid of Comparative Example 4. As presented in Table 4, the CSS measurement of the magnetic disk was 11,310 times, and the CSS measurement after the heat test was 6,500 times, hence the result of durability was insufficient.

### Comparative Example 22

### <Disk Durability Test 2>

The above-described magnetic disk was produced using a lubricant including the ionic liquid of Comparative Example 5. As presented in Table 4, the CSS measurement of the magnetic disk was greater than 50,000 times, and the CSS measurement after the heat test was greater than 50,000 times, hence excellent durability was exhibited.

### Comparative Example 23

### <Disk Durability Test 2>

The above-described magnetic disk was produced using a lubricant including Z-DOL. As presented in Table 4, the CSS measurement of the magnetic disk was greater than 50,000 times, but the CSS measurement after the heat test was 12,000 times, hence durability was impaired by the heat test.

### Comparative Example 24

### <Disk Durability Test 2>

The above-described magnetic disk was produced using a lubricant including Z-TETRAOL. As presented in Table 4, the CSS measurement of the magnetic disk was greater than 50,000 times, but the CSS measurement after the heat test was 36,000 times, hence durability was impaired by the heat test.

The results of Examples 34 to 48 and Comparative Examples 18 to 24 are summarized in Table 4.

TABLE 4

| | | | | CSS durability | |
|---|---|---|---|---|---|
| | Lubricant | CSS durability | | after heating | |
| Ex. 34 | Ex. 1 | 25° C., | >50,000 | 25° C., | >50,000 |
| | | 60% RH | | 60% RH | |
| Ex. 35 | Ex. 2 | 25° C., | >50,000 | 25° C., | >50,000 |
| | | 60% RH | | 60% RH | |
| Ex. 36 | Ex. 3 | 25° C., | >50,000 | 25° C., | >50,000 |
| | | 60% RH | | 60% RH | |
| Ex. 37 | Ex. 4 | 25° C., | >50,000 | 25° C., | >50,000 |
| | | 60% RH | | 60% RH | |
| Ex. 38 | Ex. 5 | 25° C., | >50,000 | 25° C., | >50,000 |
| | | 60% RH | | 60% RH | |
| Ex. 39 | Ex. 6 | 25° C., | >50,000 | 25° C., | >50,000 |
| | | 60% RH | | 60% RH | |
| Ex. 40 | Ex. 7 | 25° C., | >50,000 | 25° C., | >50,000 |
| | | 60% RH | | 60% RH | |
| Ex. 41 | Ex. 8 | 25° C., | >50,000 | 25° C., | >50,000 |
| | | 60% RH | | 60% RH | |
| Ex. 42 | Ex. 9 | 25° C., | >50,000 | 25° C., | >50,000 |
| | | 60% RH | | 60% RH | |
| Ex. 43 | Ex. 10 | 25° C., | >50,000 | 25° C., | >50,000 |
| | | 60% RH | | 60% RH | |
| Ex. 44 | Ex. 11 | 25° C., | >50,000 | 25° C., | >50,000 |
| | | 60% RH | | 60% RH | |
| Ex. 45 | Ex. 12 | 25° C., | >50,000 | 25° C., | >50,000 |
| | | 60% RH | | 60% RH | |
| Ex. 46 | Ex. 13 | 25° C., | >50,000 | 25° C., | >50,000 |
| | | 60% RH | | 60% RH | |
| Ex. 47 | Ex. 14 | 25° C., | >50,000 | 25° C., | >50,000 |
| | | 60% RH | | 60% RH | |
| Ex. 48 | Ex. 15 | 25° C., | >50,000 | 25° C., | >50,000 |
| | | 60% RH | | 60% RH | |
| Comp. | Comp. | 25° C., | >50,000 | 25° C., | >50,000 |
| Ex. 18 | Ex. 1 | 60% RH | | 60% RH | |
| Comp. | Comp. | 25° C., | 13200 | 25° C., | 8965 |
| Ex. 19 | Ex. 2 | 60% RH | | 60% RH | |
| Comp. | Comp. | 25° C., | 12350 | 25° C., | 8700 |
| Ex. 20 | Ex. 3 | 60% RH | | 60% RH | |
| Comp. | Comp. | 25° C., | 11310 | 25° C., | 6500 |
| Ex. 21 | Ex. 4 | 60% RH | | 60% RH | |
| Comp. | Comp. | 25° C., | >50,000 | 25° C., | >50,000 |
| Ex. 22 | Ex. 5 | 60% RH | | 60% RH | |
| Comp. | Z-DOL | 25° C., | >50,000 | 25° C., | 12,000 |
| Ex. 23 | | 60% RH | | 60% RH | |
| Comp. | Z- | 25° C., | >50,000 | 25° C., | 36,000 |
| Ex. 24 | TETRAOL | 60% RH | | 60% RH | |

Next, examples where lubricants of the novel dication-based ionic liquids of Examples 1 to 15, lubricants of Comparative Examples 1 to 5, and Z-DOL and Z-TETRAOL of the commercial products are applied to magnetic tapes are described.

### Examples 49 to 63 and Comparative Examples 25 to 31

The above-described magnetic tape was produced using a lubricant including each of ionic liquids of Examples 1 to 15, ionic liquids of Comparative Examples 1 to 5, Z-DOL, and Z-TETRAOL. Then, the following measurements were performed. The results are presented in Tables 5-1 to 5-3.

Friction Coefficient of the Magnetic Tape after Shuttle Run of 100 Times

In the environment having a temperature of −5° C. or in the environment having a temperature of 40° C. and relative humidity of 90%.

Still Durability Test

In the environment having a temperature of −5° C. or in the environment having a temperature of 40° C. and relative humidity of 30%.

Shuttle Durability Test

In the environment having a temperature of −5° C. or in the environment having a temperature of 40° C. and relative humidity of 90%.

Friction Coefficient of the Magnetic Tape after Shuttle Run of 100 Times after the Heat Test

In the environment having a temperature of −5° C. or in the environment having a temperature of 40° C. and relative humidity of 90%.

Still Durability Test after the Heat Test

In the environment having a temperature of −5° C. or in the environment having a temperature of 40° C. and relative humidity of 30%.

Shuttle Durability Test after the Heat Test

In the environment having a temperature of −5° C. or in the environment having a temperature of 40° C. and relative humidity of 90%.

TABLE 5-1

| | | | | | | | | Friction | | | | | |
|---|---|---|---|---|---|---|---|---|---|---|---|---|---|
| | | | | | | | | coefficient | | Still | | Shuttle | |
| | | Friction | | | | | | after | | durability | | durability | |
| | | coefficient | | Still | | Shuttle | | 100 runs | | after | | after | |
| | Com- | after | | durability/ | | durability/ | | after | | heating/ | | heating/ | |
| | pound | 100 runs | | min | | time | | heating | | min | | time | |
| Ex. 49 | Ex. 1 | −5° C. | 0.18 | −5° C. | >60 | −5° C. | >200 | −5° C. | 0.19 | −5° C. | >60 | −5° C. | >200 |
| | | 40° C., | 0.19 | 40° C., | >60 | 40° C., | >200 | 40° C., | 0.19 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 50 | Ex. 2 | −5° C. | 0.19 | −5° C. | >60 | −5° C. | >200 | −5° C. | 0.19 | −5° C. | >60 | −5° C. | >200 |
| | | 40° C., | 0.20 | 40° C., | >60 | 40° C., | >200 | 40° C., | 0.21 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 51 | Ex. 3 | −5° C. | 0.20 | −5° C. | >60 | −5° C. | >200 | −5° C. | 0.20 | −5° C. | >60 | −5° C. | >200 |
| | | 40° C., | 0.21 | 40° C., | >60 | 40° C., | >200 | 40° C., | 0.22 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 52 | Ex. 4 | −5° C. | 0.17 | −5° C. | >60 | −5° C. | >200 | −5° C. | 0.18 | −5° C. | >60 | −5° C. | >200 |
| | | 40° C., | 0.18 | 40° C., | >60 | 40° C., | >200 | 40° C., | 0.18 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 53 | Ex. 5 | −5° C. | 0.18 | −5° C. | >60 | −5° C. | >200 | −5° C. | 0.19 | −5° C. | >60 | −5° C. | >200 |
| | | 40° C., | 0.18 | 40° C., | >60 | 40° C., | >200 | 40° C., | 0.19 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 54 | Ex. 6 | −5° C. | 0.18 | −5° C. | >60 | −5° C. | >200 | −5° C. | 0.20 | −5° C. | >60 | −5° C. | >200 |
| | | 40° C., | 0.19 | 40° C., | >60 | 40° C., | >200 | 40° C., | 0.20 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 55 | Ex. 7 | −5° C. | 0.18 | −5° C. | >60 | −5° C. | >200 | −5° C. | 0.19 | −5° C. | >60 | −5° C. | >200 |
| | | 40° C., | 0.19 | 40° C., | >60 | 40° C., | >200 | 40° C., | 0.19 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 56 | Ex. 8 | −5° C. | 0.19 | −5° C. | >60 | −5° C. | >200 | −5° C. | 0.19 | −5° C. | >60 | −5° C. | >200 |
| | | 40° C., | 0.20 | 40° C., | >60 | 40° C., | >200 | 40° C., | 0.21 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 57 | Ex. 9 | −5° C. | 0.20 | −5° C. | >60 | −5° C. | >200 | −5° C. | 0.20 | −5° C. | >60 | −5° C. | >200 |
| | | 40° C., | 0.21 | 40° C., | >60 | 40° C., | >200 | 40° C., | 0.22 | 40° C., | >60 | 40° C., | >200 |
| | | 90% | | 30% | | 90% | | 90% | | 30% | | 90% | |
| | | RH | | RH | | RH | | RH | | RH | | RH | |

TABLE 5-2

| | | | | | | | | Friction | | | | | |
|---|---|---|---|---|---|---|---|---|---|---|---|---|---|
| | | | | | | | | coefficient | | Still | | Shuttle | |
| | | Friction | | | | | | after | | durability | | durability | |
| | | coefficient | | Still | | Shuttle | | 100 runs | | after | | after | |
| | Com- | after | | durability/ | | durability/ | | after | | heating/ | | heating/ | |
| | pound | 100 runs | | min | | time | | heating | | min | | time | |
| Ex. 58 | Ex. 10 | −5° C. | 0.19 | −5° C. | >60 | −5° C. | >200 | −5° C. | 0.20 | −5° C. | >60 | −5° C. | >200 |
| | | 40° C., | 0.20 | 40° C., | >60 | 40° C., | >200 | 40° C., | 0.20 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 59 | Ex. 11 | −5° C. | 0.20 | −5° C. | >60 | −5° C. | >200 | −5° C. | 0.21 | −5° C. | >60 | −5° C. | >200 |
| | | 40° C., | 0.21 | 40° C., | >60 | 40° C., | >200 | 40° C., | 0.21 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 60 | Ex. 12 | −5° C. | 0.18 | −5° C. | >60 | −5° C. | >200 | −5° C. | 0.19 | −5° C. | >60 | −5° C. | >200 |
| | | 40° C., | 0.19 | 40° C., | >60 | 40° C., | >200 | 40° C., | 0.19 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 61 | Ex. 13 | −5° C. | 0.18 | −5° C. | >60 | −5° C. | >200 | −5° C. | 0.18 | −5° C. | >60 | −5° C. | >200 |
| | | 40° C., | 0.18 | 40° C., | >60 | 40° C., | >200 | 40° C., | 0.19 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 62 | Ex. 14 | −5° C. | 0.20 | −5° C. | >60 | −5° C. | >200 | −5° C. | 0.20 | −5° C. | >60 | −5° C. | >200 |
| | | 40° C., | 0.21 | 40° C., | >60 | 40° C., | >200 | 40° C., | 0.21 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 63 | Ex. 15 | −5° C. | 0.21 | −5° C. | >60 | −5° C. | >200 | −5° C. | 0.21 | −5° C. | >60 | −5° C. | >200 |
| | | 40° C., | 0.21 | 40° C., | >60 | 40° C., | >200 | 40° C., | 0.22 | 40° C., | >60 | 40° C., | >200 |
| | | 90% | | 30% | | 90% | | 90% | | 30% | | 90% | |
| | | RH | | RH | | RH | | RH | | RH | | RH | |

TABLE 5-3

| | | | | | | | | Friction | | | | | |
|---|---|---|---|---|---|---|---|---|---|---|---|---|---|
| | | | | | | | | coefficient | | Still | | Shuttle | |
| | | Friction | | | | | | after | | durability | | durability | |
| | | coefficient | | Still | | Shuttle | | 100 runs | | after | | after | |
| | Com- | after | | durability/ | | durability/ | | after | | heating/ | | heating/ | |
| | pound | 100 runs | | min | | time | | heating | | min | | time | |
| Comp. | Comp. | −5° C. | 0.22 | −5° C. | >60 | −5° C. | >200 | −5° C. | 0.25 | −5° C. | >60 | −5° C. | >200 |
| Ex. 25 | Ex. 1 | 40° C., | 0.25 | 40° C., | >60 | 40° C., | >200 | 40° C., | 0.27 | 40° C., | >60 | 40° C. | >200 |
| | | 90% | | 30% | | 90% | | 90% | | 30% | | 90% | |
| | | RH | | RH | | RH | | RH | | RH | | RH | |
| Comp. | Comp. | −5° C. | 0.32 | −5° C. | 11 | −5° C. | 45 | −5° C. | 0.34 | −5° C. | 12 | −5° C. | 46 |
| Ex. 26 | Ex. 2 | 40° C., | 0.35 | 40° C., | 16 | 40° C., | 65 | 40° C., | 0.36 | 40° C., | 15 | 40° C., | 58 |
| | | 90% | | 30% | | 90% | | 90% | | 30% | | 90% | |
| | | RH | | RH | | RH | | RH | | RH | | RH | |
| Comp. | Comp. | −5° C. | 0.33 | −5° C. | 13 | −5° C. | 65 | −5° C. | 0.34 | −5° C. | 10 | −5° C. | 55 |
| Ex. 27 | Ex. 3 | 40° C., | 0.36 | 40° C., | 18 | 40° C., | 71 | 40° C., | 0.37 | 40° C., | 11 | 40° C., | 65 |
| | | 90% | | 30% | | 90% | | 90% | | 30% | | 90% | |
| | | RH | | RH | | RH | | RH | | RH | | RH | |
| Comp. | Comp. | −5° C. | 0.35 | −5° C. | 15 | −5° C. | 54 | −5° C. | 0.35 | −5° C. | 14 | −5° C. | 45 |
| Ex. 28 | Ex. 4 | 40° C., | 0.36 | 40° C., | 16 | 40° C., | 75 | 40° C., | 0.37 | 40° C., | 13 | 40° C., | 61 |
| | | 90% | | 30% | | 90% | | 90% | | 30% | | 90% | |
| | | RH | | RH | | RH | | RH | | RH | | RH | |
| Comp. | Comp. | −5° C. | 0.22 | −5° C. | >60 | −5° C. | >200 | −5° C. | 0.24 | −5° C. | >60 | −5° C. | >200 |
| Ex. 29 | Ex. 5 | 40° C., | 0.26 | 40° C., | >60 | 40° C. , | >200 | 40° C., | 0.26 | 40° C., | >60 | 40° C. | >200 |
| | | 90% | | 30% | | 90% | | 90% | | 30% | | 90% | |
| | | RH | | RH | | RH | | RH | | RH | | RH | |
| Comp. | Z-DOL | −5° C. | 0.25 | −5° C. | 12 | −5° C. | 59 | −5° C. | 0.32 | −5° C. | 12 | −5° C. | 46 |
| Ex. 30 | | 40° C., | 0.30 | 40° C., | 48 | 40° C., | 124 | 40° C., | 0.35 | 40° C. | 15 | 40° C., | 58 |
| | | 90% | | 30% | | 90% | | 90% | | 30% | | 90% | |
| | | RH | | RH | | RH | | RH | | RH | | RH | |
| Comp. | Z- | −5° C. | 0.22 | −5° C. | 25 | −5° C. | 65 | −5° C. | 0.28 | −5° C. | 23 | −5° C. | 55 |
| Ex. 31 | TETRAOL | 40° C., | 0.26 | 40° C., | 35 | 40° C., | 156 | 40° C., | 0.32 | 40° C., | 31 | 40° C., | 126 |
| | | 90% | | 30% | | 90% | | 90% | | 30% | | 90% | |
| | | RH | | RH | | RH | | RH | | RH | | RH | |

In the tables above, “>60” of the still durability means greater than 60 minutes. In the tables above, “>200” of the shuttle durability means greater than 200 times.

It was found that the magnetic tapes, to which the lubricants including the ionic liquids of Examples 1 to 15, respectively, were applied, had excellent antifriction properties, still durability, and shuttle durability.

It was found that the magnetic tape to which the lubricant including the ionic liquid of Comparative Example 1 was applied had excellent antifriction properties, still durability, and shuttle durability. The lubricant of Comparative Example 1 exhibits excellent the magnetic tape durability.

It was found that the magnetic tapes, to which lubricants including the ionic liquids of Comparative Examples 2 to 4, respectively, were applied, were not satisfactory in terms of antifriction properties, still durability, and shuttle durability.

It was found that the magnetic tape to which Z-DOL was applied had significant deteriorations in still durability and shuttle durability.

It was found that the magnetic tape to which Z-TETRAOL was applied had significant deteriorations in still durability and shuttle durability.

It was found from Tables 3 to 5 that excellent heat resistance and durability of the magnetic tape and the magnetic disk were obtained by using the ionic liquid-based lubricant including a conjugate base and a conjugate acid including 2 or more cations in a molecular of the conjugate acid, where pKa of an acid that was a base of the conjugate base in acetonitrile was 10 or less.

As clear from the descriptions above, the ionic liquid-based lubricant including a conjugate base and a conjugate acid including 2 or more cations in a molecular of the conjugate acid, where pKa of an acid that was a base of the conjugate base in acetonitrile was 10 or less, has high decomposition temperature and 5%, 10%, and 20% weight reduction temperatures, and excellent thermal stability. Under high temperature conditions, moreover, excellent lubricity can be maintained compared to conventional perfluoropolyether, and moreover, the lubricity can be maintained over a long period. Accordingly, a magnetic recording medium using the lubricant including the ionic liquid can obtain extremely excellent running performances, antifriction properties, and durability.

### REFERENCE SIGNS LIST

11 substrate
12 undercoat layer
13 magnetic layer
14 protective carbon layer
15 lubricant layer
21 substrate
22 magnetic layer
23 protective carbon layer
24 lubricant layer
25 back-coating layer

1. (canceled)

2. (canceled)

3. (canceled)

4. (canceled)

5. (canceled)

6. (canceled)

7. (canceled)

8. A lubricant comprising:
an ionic liquid including a conjugate base and conjugate acid,
wherein an acid that is the source of the conjugate base has a pKa in acetonitrile of 10 or less, and
wherein the ionic liquid is represented by General Formula (4) below: 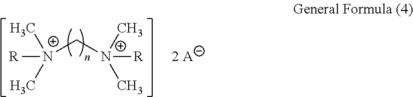
where, in General Formula (4), each R is independently a group including a straight-chain hydrocarbon group having 6 or more carbon atoms, A⁻ is the conjugate base, and n is 1 or greater but 20 or less.

9. The lubricant according to claim 8,
wherein the ionic liquid is represented by General Formula (4-1) below, General Formula (4-2) below, General Formula (4-3) below, or General Formula (4-4) below: 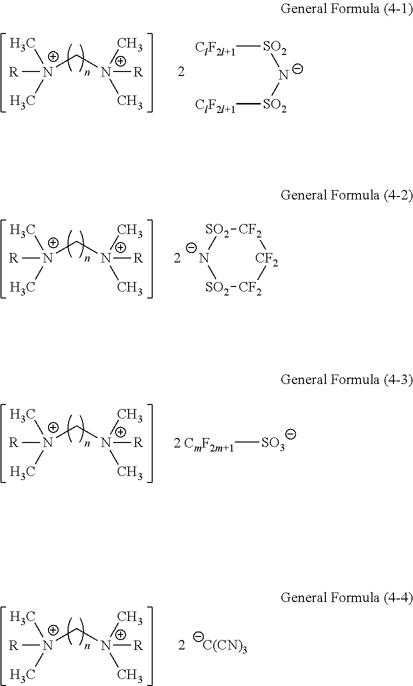
where, in General Formula (4-1), each R is independently a group including a straight-chain hydrocarbon group having 6 or more carbon atoms, n is 1 or greater but 20 or less, and l is 1 or greater but 12 or less,
in General Formula (4-2), each R is independently a group including a straight-chain hydrocarbon group having 6 or more carbon atoms, and n is 1 or greater but 20 or less,
in General Formula (4-3), each R is independently a group including a straight-chain hydrocarbon group having 6 or more carbon atoms, n is 1 or greater but 20 or less, and m is 0 or greater but 12 or less, and
in General Formula (4-4), each R is independently a group including a straight-chain hydrocarbon group having 6 or more carbon atoms, and n is 1 or greater but 20 or less.

10. A magnetic recording medium comprising:
a non-magnetic support;
a magnetic layer disposed on or above the non-magnetic support; and
the lubricant according to claim 8 disposed on or above the magnetic layer.

11. (canceled)

12. (canceled)

13. (canceled)

14. (canceled)

15. (canceled)

16. (canceled)

17. (canceled)

18. An ionic liquid comprising:
a conjugate base; and
a conjugate acid,
wherein an acid that is a source of the conjugate base has a pKa in acetonitrile of 10 or less, and
wherein the ionic liquid is represented by General Formula (4) below: 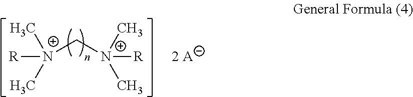
where, in General Formula (4), each R is independently a group including a straight-chain hydrocarbon group having 6 or more carbon atoms, A⁻ is the conjugate base, and n is 1 or greater but 20 or less.

19. The ionic liquid according to claim 18,
wherein the ionic liquid is represented by General Formula (4-1) below, General Formula (4-2) below, General Formula (4-3) below, or General Formula (4-4) below: 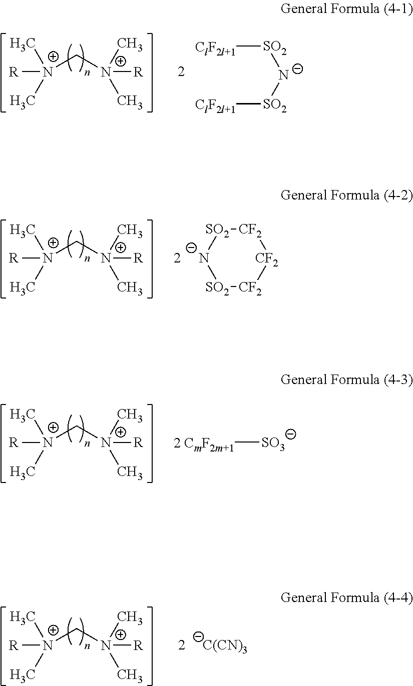
where, in General Formula (4-1), each R is independently a group including a straight-chain hydrocarbon group having 6 or more carbon atoms, n is 1 or greater but 20 or less, and l is 1 or greater but 12 or less,
in General Formula (4-2), each R is independently a group including a straight-chain hydrocarbon group having 6 or more carbon atoms, and n is 1 or greater but 20 or less,
in General Formula (4-3), each R is independently a group including a straight-chain hydrocarbon group having 6 or more carbon atoms, n is 1 or greater but 20 or less, and m is 0 or greater but 12 or less, and
in General Formula (4-4), each R is independently a group including a straight-chain hydrocarbon group having 6 or more carbon atoms, and n is 1 or greater but 20 or less.
